(19) United States
(12) Patent Application Publication
(10) Pub. No.: US 2025/0378935 A1
(43) Pub. Date: Dec. 11, 2025

(54) SYSTEM AND METHOD FOR DETERMINING DIETARY IMPACTS ON ENVIRONMENTAL MEASURES AND ASSESSING DIET QUALITY IN VIEW OF CULTURAL DIETARY DIFFERENCES

(71) Applicant: Diet ID, Inc., Detroit, MI (US)

(72) Inventors: David L. Katz, Hamden, CT (US); Lauren Q. Rhee, Fulton, MD (US); Marie Janiszewski, Detriot, MI (US); Dina L. Aronson, Bloomfield, NJ (US); Gidon Eshel, Detroit, MI (US); Martin C. Heller, Detroit, MI (US); Emily Barrett, Detroit, MI (US); Austin Cameron, Louisville, KY (US)

(21) Appl. No.: 19/309,367

(22) Filed: Aug. 25, 2025

Related U.S. Application Data
(63) Continuation-in-part of application No. 18/814,914, filed on Aug. 26, 2024.
Application No. 18/814,914 is a continuation of application No. 17/352,166, filed on Jun. 18, 2021, now Pat. No. 12,073,935.
Application No. 17/352,166 is a continuation-in-part of application No. PCT/US2019/067909, filed on Dec. 20, 2019.
(60) Provisional application No. 62/782,773, filed on Dec. 20, 2018.
(60) Provisional application No. 63/687,379, filed on Aug. 27, 2024.
(60) Provisional application No. 63/692,892, filed on Sep. 10, 2024.

(51) Int. Cl. G16H 20/60; G06F 16/432; G06F 16/50; G06F 16/51; G06F 16/532; G06F 16/535; G06F 16/538; G06V 20/68; G09B 5/02; G09B 19/00

(52) U.S. Cl. CPC G16H 20/60; G06F 16/434; G06F 16/50; G06F 16/51; G06F 16/532; G06F 16/535; G06F 16/538; G06V 20/68; G09B 5/02; G09B 19/0092; G06T 2207/30128; Y10S 128/921

(57) ABSTRACT

Systems and methods for determining dietary impacts on environmental measures and for identifying and assessing the quality of an individual's diet in view of cultural/prevailing dietary variance are discussed.

### RELATED APPLICATIONS

This application claims the benefit of, and priority to, U.S. Provisional Application No. 63/687,379 entitled “System and Method for Determining Dietary Impacts on Environmental Measures”, filed Aug. 27, 2024, and to U.S. Provisional Application No. 63/692,892 entitled “System and Method for Performing Dietary Quality Assessment in View of Cultural Diet Differences”, filed Sep. 10, 2024. This application is also a continuation-in-part of U.S. patent application Ser. No. 18/814,914, entitled “System and Method for Assessing Diet Quality Via Photo Navigation Using Dietary Fingerprints”, filed Aug. 26, 2024, which is a continuation of U.S. patent application Ser. No. 17/352,166, now U.S. Pat. No. 12,073,935 entitled “Systems and Methods for Diet Quality Photo Navigation Utilizing Dietary Fingerprints for Diet Assessment”, filed Jun. 18, 2021, which is a continuation-in-part of PCT Application No. PCT/US2019/067909, filed Dec. 20, 2019, which claimed the benefit of, and priority to U.S. Provisional Patent Application No. 62/782,773 filed Dec. 20, 2018. The contents of all of the above applications being incorporated herein by reference in their entirety.

### BACKGROUND

Diet quality does not merely influence human health but also stands out as the single leading predictor of risk for both premature death from all causes as well as the risk of chronic disease in the modern world. Accordingly, a number of attempts have been made to provide objective standards of diet quality. For example, the Healthy Eating Index (HEI) and the related Alternative Healthy Index (AHI) are the most widely used and robustly validated measures of overall diet quality. Those dietary patterns associated with optimal human health tend to correspond with dietary patterns associated with optimal planetary health. Similarly, dietary patterns associated with less desirable human health outcomes tend to have a greater negative impact on planetary health. The environmental impacts of the growth and consumption of different types of foods, which include impacts on land use, water use, nitrogen inputs and greenhouse gas emissions, are substantial, and vary widely. Public databases exist to quantify the separate environmental impacts of the production/growth and consumption of different types of individual foods/ingredients/dietary elements.

### BRIEF SUMMARY

Embodiments of the present invention provide techniques for determining and quantifying dietary impacts on environmental measures (DIEM) for an individual's overall dietary pattern. More particularly, techniques for identifying an individual's current dietary plan and associating it in real-time with its corresponding quantified environmental impact are provided. Further, embodiments provide a range of alternative diet goal suggestions to the individual to both improve the individual's diet from a health standpoint while at the same time offering options to lessen environmental impacts. Embodiments further provide suggested steps for transitioning from a current diet to a suggested diet. Additionally, leveraged at a population scale, embodiments can provide important information regarding impacts on planetary and environmental health, and provide a basis for favorable shifts in same. Further, embodiments also provide an assessment of a user's current diet that includes a value indicative of diet quality and adjust that value to account for the inclusion or exclusion of discretionary dietary elements stemming from variable practices with regard to dietary patterns, and to cultural dietary variations.

In one embodiment, a computing device-implemented method for determining dietary impact on environmental measures with a computing device that includes at least one processor, includes providing a database of images depicting dietary elements representative of a multi-day dietary intake pattern, the dietary elements being at least one of food and beverages. The method also includes receiving, at a server, a request from a client device for images for use in diet determination and providing, in response to the request, a sequence of image vignettes for photo navigation generated from the database of images. Each image vignette is generated from initial images associated a multi-day dietary intake pattern and includes a subset of dietary elements depicted in the initial images, the subset containing fewer images than depicted in the initial image. The method additionally determines a target diet including a diet type and diet quality for a user based on a sequence of photo navigation choices of dietary elements made by the user from among the provided sequence of image vignettes, the determined target diet including multiple dietary elements. Further the method determines different types of environmental impact scores for each of the dietary elements in the target diet, each environmental impact score respectively associated with a different type of environmental impact and determined using one or more environmental impact databases. The method also generates a cumulative environmental impact score for each dietary element in the determined diet based on each of the types of environmental impact scores for that dietary element and determines a cumulative environmental impact score for the target diet based on the generated cumulative environmental impact scores for the dietary elements in the target diet and a proportional contribution of each dietary element in terms of calories to the target diet.

In another embodiment, a system for determining dietary impact on environmental measures includes one or more databases of images depicting dietary elements representative of a multi-day dietary intake pattern, the dietary elements being at least one of food and beverages. The system also includes one or more environmental impact databases holding measurements of different types of environmental impacts associated with the dietary elements. The system further includes a server configured to receive a request from a client device for images for use in diet determination, wherein in response to the request a sequence of image vignettes for photo navigation generated from the database of images is provided. Each image vignette is generated from initial images associated with a multi-day dietary intake pattern and includes a subset of dietary elements depicted in the initial images, the subset containing fewer images than depicted in the initial images. A target diet that includes a diet type and diet quality is determined for a user based on a sequence of photo navigation choices of dietary elements made by the user from among the provided sequence of image vignettes, the determined target diet including multiple dietary elements. Different types of environmental impact scores for each of the dietary elements in the target diet are determined with each environmental impact score respectively associated with a different type of environmental impact and determined using the one or more environmental impact databases. A cumulative environmental impact score for each dietary element in the determined diet is also determined based on each of the types of environmental impact scores for that dietary element. Additionally, a cumulative environmental impact score for the target diet is determined based on the generated cumulative environmental impact scores for the dietary elements in the target diet and a proportional contribution of each dietary element in terms of calories to the target diet.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1 depicts an illustration of a diet grid of diet types N and levels of diet quality X;

FIG. 2 depicts a system including an environment for managing processes in some embodiments;

FIG. 3 depicts an exemplary system for generating image vignettes;

FIG. 4 depicts an exemplary sequence of steps followed by an embodiment of the present invention to determine an environmental impact of a user's target diet.

FIG. 5 depicts an exemplary environment suitable for practicing one or more embodiments of the present invention;

FIG. 6 depicts an exemplary sequence of steps followed by an embodiment of the present invention to determine an impact of cultural dietary differences on an individual's target diet; and

FIG. 7 depicts an exemplary environment suitable for practicing one or more embodiments of the present invention.

### DETAILED DESCRIPTION

Databases exist to quantify the separate environmental impacts of the production and consumption of different types of foods, ingredients, and dietary elements, but aggregate environmental impact scores for foods across multiple component measures (e.g., land use, water utilization, greenhouse gas emissions, etc.) are less readily available, and quantified environmental impact scores for an entire assembly of food choices populating an entire dietary pattern—whether for individual or group-are not established. The ability to compare the environmental footprint of diverse dietary options, or to compare current to prospective diet—is not supported by extant means. Utility to consumers is further limited by lack of guidance at the level of overall dietary pattern of the environmental footprints of diets varying by both type and quality and the lack of the ability to adjust dietary assessment based on the inclusion or exclusion of discretionary dietary components stemming from cultural dietary differences and variations in prevailing dietary patterns as practiced by populations. Currently there is no established method to identify a given individual's current diet or personalized goal diet, and immediately assign a standardized, quantitative measure of environmental impact. By providing such information in real time and contextualizing it to make it actionable across a range of options, embodiments of the present invention enable an individual to make diet choices with both personal health and planetary health/environmental impact in mind. Further embodiments enable the assessed value of the user's current diet to be adjusted to take into account the inclusion or exclusion of discretionary components (i.e., food groups) from the diet based on cultural dietary differences.

Embodiments of the present invention determine the quantitative, overall environmental ‘footprint’ of dietary intake patterns building on recently developed techniques for performing diet mapping as well as techniques for dietary fingerprinting enabling dietary pattern information to be conveyed more efficiently to an individual looking to assess and improve their diet. In recent years, systems and methods for diet mapping and the use of a diet map for dietary assessment have been described in U.S. patent application Ser. No. 16/614,675, the contents of which are incorporated herein by reference in their entirety. Further, systems and methods for dietary fingerprinting have been described in U.S. patent application Ser. No. 17/352,166, the contents of which are also incorporated herein by reference in their entirety. Prior to describing embodiments of the present invention for determining dietary impacts on environmental measures (DIEM scores) and performing dietary assessment in light of cultural and other extant dietary differences as they relate to the inclusion or exclusion of select discretionary food groups (e.g., dairy), exemplary systems for providing diet mapping, diet maps and diet fingerprinting that may be utilized by embodiments to determine DIEM scores and assist in performing dietary assessment in light of cultural/prevailing dietary differences are first described.

### Exemplary Diet Mapping System

As described in U.S. patent application Ser. No. 17/739,593, levels of diet quality can be translated into photographic representations of a dietary pattern, by steps including:
a) using a diet quality measure to identify multiple dietary patterns that each represent a level of diet quality for a period of time;
b) assigning a dietary score to each of the dietary patterns;
c) converting the dietary patterns into representative dietary patterns; and
d) converting the representative dietary patterns into composite images,
wherein each composite image depicts a photographic representation of

the dietary patterns for the period of time.

Thus, using this methodology, a library of composite images can be created in which each composite depicts a unique inventory of proportions of foods, ingredients, dishes, and meals, representative of a particular diet quality level X of a particular diet type N for a period of time.

The dietary patterns may include a number of typical dietary patterns for a given population, considering “poor,” “good,” “better,” and “best” diets for the given population. Thus, using this methodology, it is possible to map differences in diets using common coordinates to create desired gradients within those diets.

It has also been determined that unique differences and characteristics of diet in terms of environmental quality or environmental sustainability can be mapped in a similar manner as characteristics and traits that distinguish types of diet and differences in levels of quality in a particular diet. Thus it is possible to quantify differences in diet quality based on health and to also quantify difference in diet quality based on environmental impacts of the measure of diet quality.

In one example, a life cycle assessment may be performed of representative traits of a particular diet type. As discussed above a life cycle assessment (LCA) can demonstrate how the food, and/or the refining of such food affects the environment, from planting and harvesting of the food, transportation, and refining of the food and until the food becomes waste or is recycled or composted.

Life cycle assessment can be evaluated by determining one or more of the following:
1) Acidification;
2) Eutrophication;
3) Global warming potential;
4) Photochemical smog; and
5) Land use

Thus, diet types N have each been mapped in terms of kind and objective quality measures to distinguish X levels of diet quality within each diet type N.

These levels of diet quality X of diet types N are translated into representative multi-day meal plans that highlight unique distinguishing characteristics of the level of diet X of the diet type N. This information is then converted into an inventory of foods that are translated into food images and these food images are transformed into composite images that represent food intake over a multi-day period for each level of diet quality X of each diet type N.

Further, a method of quantifying and mapping diet quality has been developed where the method includes the steps of:
a) identifying a dietary pattern with a meaningful prevalence in a geographical region of interest;
b) cataloging defining and exclusive attributes of the dietary pattern;
c) prioritizing diet quality measures validated against health outcomes;
d) establishing features of a selected diet and the relative variance within the features;
e) identifying a sequence of dietary variants representing the relative variable within the features; and
f) determining an appropriate degree of discrimination required to represent realistic variation in the dietary pattern.

The first step to quantify and map diet quality is to select a diet type N for analysis, which may be one of a number of diet types that represent common diet types of a typical user in a geographical area of interest. For example, the procedure may involve the step of identifying a dietary pattern with a minimal, meaningful prevalence (i.e., greater than or equal to about 5%) in the geographic region of interest. This step is repeated until about 95% of the general population of interest is represented. The dietary pattern may be one such as, for example, vegan, Southwestern, etc. Prevalence is preferably based on published literature/epidemiological analysis when possible and, if necessary, with expert opinion/personal experience as a contingency, The resulting product is a general diet type N that can be used to populate a ROW in the DQPN map.

The next step is to establish an exclusive, operational definition of the diet type N. For example, the procedure may involve cataloguing the defining and exclusive attributes of the diet type in question. The definition and exclusive attributes of the diet in question may be one such as, for example, a vegan diet consisting of a plant food diet exclusive of all meat, fish, eggs, dairy and seafood. The resulting product is a specific and exclusive diet type N, suitable for mapping in a DQPN ROW.

The next step is to identify a suitable measure of objective diet quality applicable to the diet type N. As one example, the procedure may involve prioritizing diet quality measures to optimize health and/or to minimize adverse environmental impacts to establish appropriate stratification based on objective quality, for example, correspondence with health outcomes, rather than fidelity to the diet principles, per se. The resulting product is an applicable diet quality measure for a given ROW of the map, wherein each cell in the row of this map represents a level of diet quality X of the diet type N.

The next step in the process is to address ‘adherence to type’ as warranted. In one example, the procedure involves establishing the salient features of a given diet type N and the relevant variance in them that account for adherence to that diet type N with greater or lesser fidelity. This step can be qualitative and subjective, based on a validated metric, or rely on Principal Component Analysis (PCA) or a related method. The differentiation among diet TYPES and the fidelity to given TYPE across the tiers of objectively measured quality are informed by Principal Component Analysis. Similarly, the mapping of PDDCs is informed by this same method for delineating the salient and differentiating attributes of a given diet type, and in particular, those most reliably associated with given health effects.

For certain diet types, such as ‘low carb’, there may be poor correlation between adherence to the principles of the diet and objective measures of diet quality. In such cases, the two may be addressed sequentially; first by establishing tiers of ‘fidelity’ to the diet principles. To do this, the defining attributes of the diet that account for its application with varying degrees of fidelity should be catalogued. So, for instance: high-fidelity to a low-carb diet would exclude more carbohydrate sources (grains, legumes, etc.) than a low-fidelity version of the same diet. If necessary and warranted, Principal Component Analysis may be considered to establish a graded sequence of ‘fidelity to type’ tiers. The resulting product is a principal of determinants of fidelity to diet type, and optionally, but preferably, a graded sequence of diet prototypes representing variable levels of fidelity and/or adherence.

The next step in the process is to stratify by score or by ‘principal determinants’ of score. In this instance, the procedure involves a sequence of dietary variants representing fidelity/adherence that are compared to the principal determinants of diet quality in a preferred, objective metric, such as, the AHEI, to determine if ‘fidelity’ and ‘quality’ are, for this given diet, positively correlated, inversely correlated, or other. To illustrate: the ‘operational’ definition of a Paleo diet might emphasize minimally processed meats, vegetables, fruits, nuts, and seeds without grains, legumes or dairy. Objective measures of diet quality might pertain poorly to assessments of the Paleo diet, but they translate into higher scores with a rising intake of vegetables, fruits, nuts and seeds; generally higher scores with fish intake than with meat intake; and generally higher scores with unprocessed as compared to processed meat intake.

Accordingly, by way of example, the Paleo diet could be stratified in accord with these principal determinants of objective diet quality measures as follows:
1. The lowest quality tier has the most meat, and the most processed meat, relative to fish, vegetables, fruits, nuts, and seeds;
2. The next higher tier has an emphasis on meat, but not processed meat;
3. The next higher tier has an emphasis on meat and fish;
4. The next higher tier has an emphasis on game and fish;
5. The next higher tier has an emphasis on fish;
6. The highest tier has the greatest emphasis on a variety of vegetables and fruits, making up the bulk of calories, with a lesser portion of the diet from fish and game.

Thus, in the case of the Paleo diet, the determination might be made that ‘fidelity’ and ‘quality’ are neither directly nor inversely correlated, since a high-fidelity, plant-predominant Paleo diet would generate a higher quality score than a comparably high-fidelity, but more meat-predominant version of the same diet. The key elements of diet stratification thus involve one of two methods: either stratify directly on the basis of objective quality scores or translate the objective quality scores into principal determinants and stratify based on the ‘subjective’ alignment of variants of a given diet with those principal determinants, and then formally score that sequence of diets to corroborate directional correctness. The resulting product is a stratified sequence of variants of the diet in question, arranged from lower quality to the left, higher quality to the right. The final product may or may not be arranged in graded order of ‘fidelity.’

The next step in the process is to estimate “ideal” stratification. In this instance, the procedure is to determine the appropriate degree of discrimination, such as, the number of quality tiers, needed to represent realistic variation in the practice of the diet type to represent levels of diet quality X. This can be obtained experience and professional judgment, as well as review of representative dietary intake assessment instructions from suitable populations. The goal is to have enough tiers of quality (levels of diet quality X) to develop images that closely approximate the diet of a given, real-world consumer and to avoid the need for excessive tiers that add clutter without clarity. The resulting product in a graded sequence of dietary variants, with an ideal number of tiers is specified.

The next step in the process involves establishing numerical “scores” for relevant tiers. In this instance, the procedure involves a number of tiers which are selected for a given diet that will determine the score ranges from the preferred metric, such as AHEI, to produce the maximal separation of dietary variants across the range from lowest to highest quality. The tiers should be situated symmetrically, for example, if there are 15 tiers, they should be spaced evenly across the expanse of 5 quintiles. The resulting product is to target scores, within narrow, specific tolerances, based on a preferred quality metric, that are used to fix the location of a given variant of a given diet in the map. Diets of a lower quality may be arranged to always appear to the left of diets of higher quality. Quality may rise in all rows from left to right.

The next step in the process is to create a menu corresponding to a cell. In this instance, the procedure involves a given cell in the DQPN map which represents a specified diet type at a target quality level, characterized by principal determinants. There are two ways to generate a menu or meal-plan based on this information: (a) assemble a prototype of foods/dishes/meals designed to hit the target score and emphasizing the principal determinants; or (b) identify several actual dietary intake assessments that correspond to the diet type and target score and hybridize those into a representative prototype. The diet prototype may be assembled and then scored, and then modified as required to move the resulting score closer to the stipulated target. Menus are preferably put together to correspond both with quality scores and with ‘real world’ eating patterns based on experience.

In this manner prototypes of actual, prevailing dietary patterns may be established. The detailed method of the menu design also includes the use of the HEI Component Score Template and/or the AHEI Component Score Template or other similar validated measure of diet quality, which involves entering the ideal component scores for each quality tier per dietary pattern. The ideal HEI-Score and/or AHEI Score range (or other similar validated measure of diet quality) can then be assigned per quality tier per dietary pattern, followed by assigning the percent goal range for macronutrients, for example, Carbs, Fat, Protein, and for other relevant nutrients, and then assigning the food group/component amounts per quality tier and specific food examples per food group/components. For example, a goal may be to aim for approximately 2500 kcal per day for up to 7 days, for each quality tier per diet type. The menu analysis may include entering food data into an established and validated nutrient analysis software program for nutrient analysis, and then adjusting specific food examples, if necessary. One such software program is the Nutrition Data System for Research (NDSR) dietary analysis program, available from the Nutrition Coordinating Center, University of Minnesota.

The final step in menu analysis is to export output data files. HEI-Scoring and/or AHEI-Scoring consists of applying the output data files (for example, into the HEI Calculation Workbook), then obtaining and reviewing the HEI-Scores and/or AHEI-Scores and adjusting menus, if necessary prior to entering the HEI-Scores and/or in AHEI-Scores in working documents, such as the Preliminary DQPN Photo Map. The resulting product is a menu or meal plan populating a given cell in the map.

In this manner, the dietary critical mass (DCM) may be established for each diet, which is the minimal quantity of food, measured in units of ‘typical daily intake,’ necessary and sufficient to represent the breadth and variety of a given diet in a composite image so that it is readily recognizable, but free of excess that does not contribute to recognition. For diets that natively include more variety, the DCM will be higher, and for diets that have little variety and routinely repeat the same, small number of foods, the DCM will be lower. Regardless of the variable DCM for each type of diet, all diets are standardized to the same number of days so that variations in quantity of food per image do not introduce unintended distractors. For diets with DCMs, rather than the extra work of inventorying additional days, the minimally adequate number of days can be inventoried, and then multiplied to produce the standardized DCM. For each diet, the DCM is analyzed for nutrient levels, including calories. In one example, the DCM is analyzed for 150 different nutrient levels.

The next step in the process is to amplify the menu to a period of time, which may be one day, several days or a full week. In this instance, the procedure involves the DQPN map showing that each image is intended to represent a typical time period of dietary intake. This may be achieved by developing seven distinct days that share the type, principal determinants, and quality score, or by developing a multi-day menu plan from the start. The time period may be represented as a mix of ingredients, dishes, and meals and need not be represented as a specific number of specific meals and snacks. If menus are assembled day-by-day, they must be expanded to represent a prototypical week. They do not need to be structured as specific meals and snacks, but rather should represent the total array of foods consumed in a ‘typical time period.’ The resulting product is a representative, time period-long meal plan corresponding to diet type, quality score, and principal determinants. While a week is a preferred period of time, the period of time may be selected to be at least one day or at least two days or at least three days or another selected time period. The analytics and specifications for each of the composite images representing each level of diet quality X of each diet type N can then be calculated.

The next step in the process is to inventory foods in specific portions. In this instance, the procedure is to specify the ingredients, dishes, and meals included in the menu plan, and establish the relative proportions of each variety of food so the quantitative representation is accurate. To prepare for photography, an exact inventory of foods and their relative quantities are necessary. The resulting product is a quantitative menu plan inventory.

The next step in the process is to specify relevant preparation details. In this instance, the procedure involves a given menu plan which may include pre-packaged food items, and meals prepared at home. The next part of this step is to establish the differential representation of these, either by showing ingredients versus packaged food, and/or by showing home-prepared meals on dishware. The composite images may differentiate between meals prepared at home and pre-prepared food consumed outside the home or at home; and such details need to be specified for each cell for appropriate representation. The resulting product is a menu plan inventory with appended description of food preparation representation.

The final step in the process is to finalize the cell description for photography and creation of the composite image. In this instance, the procedure involves establishing the final, detailed, fully characterized food inventory for styling and photography. Once the final, detailed fully characterized inventory of foods, ingredients, ingredients, dishes and meals representative of a particular diet quality level X of a particular diet type N for a period of time is styled, it is photographed to create a composite image representative of the particular diet quality level X of the particular diet type N and this step can be repeated for each diet quality level of each diet type N. The final, detailed description should translate into both a shopping list, and instructions for food prep necessary before photographing. The resulting product is a shopping list and food prep instructions.

In one instance, each row in a DQPN map will depict rising diet quality from left to right.

Each column in a DQPN map may represent movement across diet types. Animal-food predominant diets, such as, Paleo; low carb. May be at the bottom; omnivorous diets, such as Mediterranean; Flexitarian, may be in the middle; and plant predominant diets, such as, vegetarian; vegan, may be at the top. Thus, there may be a gradient from animal-food predominant to plant-food predominant from bottom to top.

The use of Principal Component Analysis, and the establishment of the principal determinants of the exclusive contents for a given cell in the map can be used as the PDDCs (principal differentiating dietary components) that characterize the ‘steps’ between a given cell and its neighbors.

Endo-PDDCs refer to the principal differentiating features among the quality tiers of a given diet across a row.

Exo-PDDCs refer to the principal differentiating features across diet types; the general direction at a given quality level may be across an expanse from animal-food predominant (bottom of map) to plant-food predominant (top of map).

Omni-PDDCs refer to the principal differentiating features that establish directionality for the entire map, for example, highly processed and animal-food predominant at the bottom left; minimally processed and plant-food predominant at the upper right.

Once the multiple dietary patterns have been identified, a dietary score may be assigned to each of the dietary patterns taking into account variations in region, culture, diet character and nutritional quality. This dietary score takes into account both the dietary quality and the environmental impacts of the particular type of diet and level of diet quality within the diet. For example, this dietary score may be an integer between 1 and 10. Thus, the lowest level of diet quality within an identified diet would be given a score of Q1 and the highest level of quality within an identified diet would be given a score of Q10. However, it is noted that this dietary score may be determined on another scale such as Q1 to QS, or Q1 to Q6, or Q1 to Q7, etc.

Furthermore, once these dietary patterns are identified, a life cycle assessment can be performed of specific exemplary foods for each diet/level of diet quality to provide an environmental score, for example, a score between 1 and 10. Thus, the level of diet quality having the most negative environmental impacts would be given a score of E1 and the level of diet quality having the least environmental impacts would be given a score of E10. It is also noted that the environmental score may not directly correlate with the dietary score. For example, even the highest quality Paleo diet, which is given a score of Q10 for Paleo diets may have more negative environmental impacts and thus be assigned a score of E6 or E7 for environmental sustainability, while the highest quality vegan diet may be given a score of Q10 for diet quality and E10 for environmental sustainability.

In order to determine the environmental score of each type of diet and level of diet quality within the type of diet, a life cycle assessment can be performed of exemplary foods within the diet. Thus, a life cycle analysis may be performed on one or more of the following:
1) meats, including red meat, pork, chicken, etc.;
2) fish, including shellfish;
3) eggs;
4) dairy, including milk, yogurt, cheese;
5) grains, including rice, wheat, etc.
6) exemplary fruits and/or vegetables, focusing on different types and growing methods;
7) packaged/processed foods, e.g., frozen dinners, crackers, cookies, etc.
8) soft drinks; and
9) other exemplary foods.

Given that diets can be mapped using common coordinates, using the process described above, it is possible to arrange diets relative to one another using these common coordinates to create desired gradients directed to minimizing environmental impacts and/or modifying diet patterns to reduce environmental impacts. For example, diets may be arranged relative to one another to create a gradient most to least likely to include meat, in combination with other objective benchmarks of diet quality.

Using an objective measure of overall environmental impact, the diets-represented in such a map can be organized to create a continuous gradient in environmental impact from most to least. In one example, the measure of overall environmental impact may be life cycle analysis as described above, or another related measure as would be known to those skilled in the art. The continuous gradient in environmental impact may include, for example, global warming potential water utilization, land use, eutrophication, acidification, photochemical smog, etc.

In general, diets of higher objective nutritional quality/better for health may correlate with diets of lesser environmental impacts. However, diets of equivalent nutritional quality for health may vary with respect to environmental impacts and vice versa. In other words, in diets of equivalent nutritional quality, a diet that is of maximum health may not be the best diet with respect to environmental impacts, especially if the diet is based on foods that are complementary foods that have a different environmental impact as determined using LCA (for example, greenhouse-grown versus field-grown tomatoes).

Using diet quality photo navigation methodology a diet quality level X of a diet type N can be represented in a composite image as fully analyzed prototypes. In this instance, the Diet Ideal becomes the ideal diet for reducing adverse environmental impacts.

In addition, the route customizing algorithm in this case becomes a coaching app to guide the user incrementally from the baseline Diet ID to a goal Diet Ideal that is chosen to minimize adverse environmental impacts.

The guidance based on the library of composite images can be further refined with specific filters. In the case of adverse environmental impacts, these filters may include, for example, organically versus conventionally grown foods, locally sourced versus transported, in season versus out-of-season, GMO versus non-GMO, by way of example and not limitation.

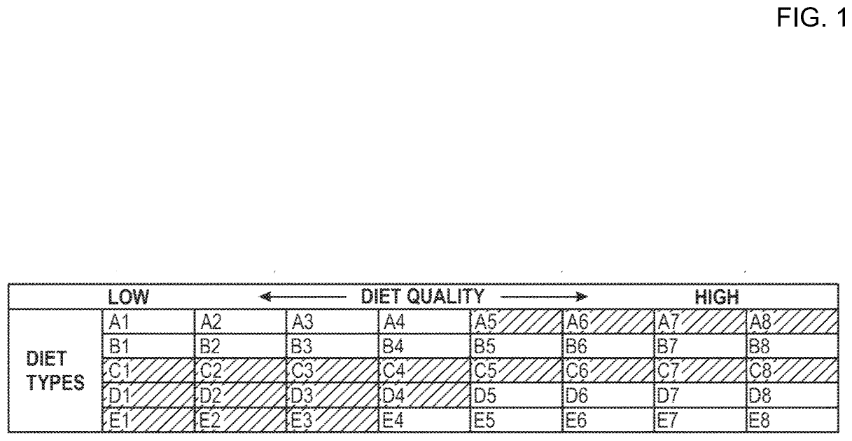

In one example, and as discussed above, the method further includes the step of selecting a number of tiers to determine a score range to produce a maximal separation of dietary variants across a range from most adverse environmental impacts to less or least adverse environmental impacts. This may overlap or coincide with dietary variants of “lowest” or “highest” dietary quality, but the intent is different. In addition, these tiers of environmental impacts can be arranged from most adverse environmental impacts lowest to highest quality, wherein the tiers are arranged symmetrically across an expanse of quintiles, wherein each tier has multiple cells as shown in FIG. 1. In another example, quality may displayed on the Y axis and type on the X axis with type sequenced based on relative preponderance of animal versus plant foods.

In addition, as described herein, each cell represents a specific diet quality level of X of a specific diet type N. The method may further include the step of generating a meal-plan corresponding to quality scores and to eating patterns based on experience. Each cell preferably depicts an image of a typical week of dietary intake for an individual or family. In one example, the image depicts a typical week of dietary intake. In another example, the image may depict a different period of time, such as one day, two days, several days, two weeks, several weeks or even one month.

The method may also include the step of establishing a meal plan based on the dietary intake, including the step of specifying food ingredients and dishes to include in the meal plan, wherein the relative proportion of each variety of food is established to produce an accurate quantitative representation.

While the DQPN route algorithm will append adjacent diet types to extend the quality scale, these may all be presented as selected diet types in a user interface that incorporates the methods described herein. So, for instance, if a user chooses a “flexitarian” diet and wants the highest possible quality and the highest quality version of that diet type migrates over to the Mediterranean diet row, then the top-quality Mediterranean diet is cross-referenced as the highest-quality flexitarian diet so that it also shows up as an option in that category.

It is further noted that the widest range of applicable diet quality levels X is not necessarily the full range. For example, the lowest quality version of a vegetarian diet would be made up of highly processed foods but would exclude meat and may also have the most adverse environmental impacts. The lowest quality version of a mixed/omnivorous diet would almost certainly score even lower, because it too would be made up of highly processed foods. But even though the “next lowest scoring diet” on the continuum might be an omnivorous selection, it would NOT make sense to present this as a choice to someone who has indicated that they are, or wish to be, vegetarian. Thus, it is important that the range of selections should be extended as far to both sides as they can be while adhering to the basic diet type (baseline or goal) indicated by the user.

The system also relates to a web-based or mobile application that can be used by consumer to evaluate the overall quality of their current diet and/or the environmental impacts of their current diet and provide affirmative steps to coach them to a higher quality diet and/or a more environmentally sustainable diet. An application program interface (API) can be used to program a computer program product that includes a graphical user interface (GUI) and that can be integrated into and delivered via an on-line platform or program, and that is viewable and executable on a computer, tablet, smart phone, or other similar device.

As will be appreciated by one skilled in the art, this system may be embodied as a system, method or computer program product. Accordingly, the system may take the form of an entirely hardware, entirely software (including firmware, resident software, micro-code, etc.) or combined software and hardware aspects that may all generally be referred to herein as a “circuit,” “module” or “system.” Furthermore, the system may take the form of a computer program product embodied in a tangible medium of expression having computer-usable program code embodied in the medium.

Different combinations of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. A non-limiting list of computer-readable medium include the following:
an electrical connection having one or more wires,
a portable computer diskette;
a hard disk,
a random-access memory (RAM),
a read-only memory (ROM),
an erasable programmable read-only memory (EPROM or Flash memory), an optical fiber,
a portable compact disc read-only memory (CDROM),
an optical storage device, and/or transmission media such as those supporting the Internet or an intranet, or a magnetic storage device.

The computer-usable or computer-readable medium can even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

As described herein, a computer-usable or computer-readable medium may be a medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using an appropriate medium, including but not limited to wireless, wireline, optical fiber cable, RF, etc.

Computer program code for carrying out operations may be written in a combination of one or more programming languages, including an object-oriented programming language such as JAVA, SMALLTALK, C++ or the like and conventional procedural programming languages, such as “C” or similar programming languages. The program code may execute entirely on the user's computer, partly on the user's computer, as a stand-alone software package, partly on the user's computer and partly on a remote computer or entirely on the remote computer or server. The remote computer may be connected to the user's computer through a type of network, including, for example, a local area network (LAN) or a wide area network (WAN), or the connection may be made to an external computer (for example, through the Internet using an Internet Service Provider).

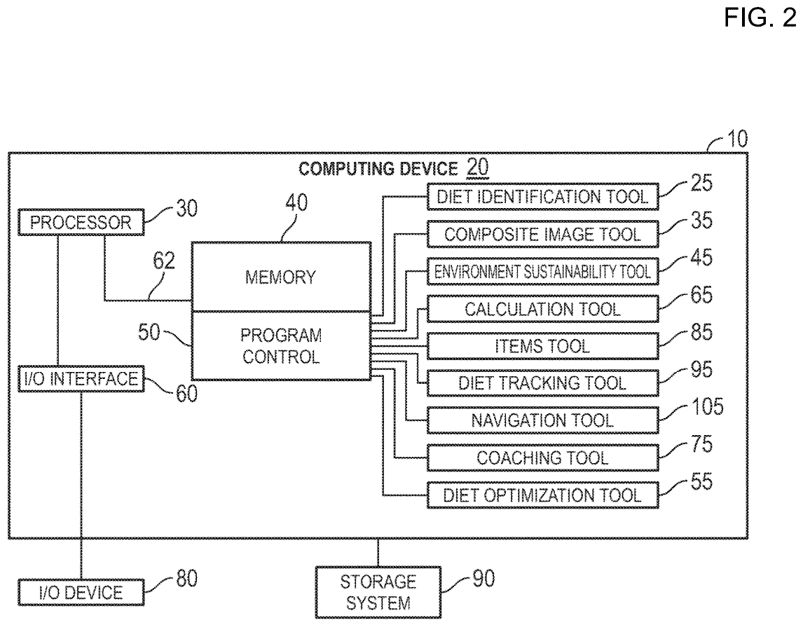

In one example and as illustrated in FIG. 2, the system includes an environment for managing the processes. The environment includes a computer infrastructure 10 that can perform the processes described herein using a computing device 20. The computing device 20 includes a Diet Identification Tool 25, a Composite Image Tool 35, An Environmental Sustainability Tool 45, a Diet Optimization Tool 55, a Calculation Tool 65, a Coaching Tool 75, a Diet Tracking Tool 95 and a Navigation Tool 105. These tools are configured to and are operable to provide a consumer with a dietary score of their current diet, a dietary score of their “Ideal” diet, and coaching/inspirational tips to move from their current diet to their ideal diet based on determined information. Thus, the processes described herein are under the control of and are controlled by program control 50.

Thus similarly to a global positioning device or App for navigating a user from point A to point B, tools are provided for a user to:
1) identify where they are (baseline diet);
2) identify where they would like to go (goal or “ideal” diet);
3) track changes over time; and
4) navigate to their destination through a series of steps.

The exemplary computing device 20 includes a processor 30, a memory 40, an input/output (I/0) interface 60, and a bus 62. The memory 40 can include local memory employed during actual execution of program code, bulk storage, and cache memories which provide temporary storage of at least some program code in order to reduce the number of times code must be retrieved from bulk storage during execution.

The computing device 20 is in communication with an external I/O device/resource 80. The I/O device 80 can interact with the computing device 20 or another device that enables the computing device 20 to communicate with one or more other computing devices using an available type of communications link. The external 1/0 device/resource 80 may be keyboards, displays, pointing devices, etc. Additionally, in some implementations, the computing device 20 includes a storage system 90.

The processor 30 executes computer program code (e.g., program control 50) processes on computer media, which is stored in memory 40 and/or storage system 90. While executing computer program code, the processor 30 can read and/or write data to/from memory 40, storage system 90, and/or I/O interface 60. The bus 62 provides a communications link between each of the components in the computing device 20.

The computing device 20 may include a general-purpose computing article of manufacture capable of executing computer program code installed thereon (such as, for example, a personal computer, server, handheld device, etc.). However, it is understood that the computing device 20 is only representative of various possible equivalent computing devices that may perform the processes described herein. To this extent, the functionality provided by the computing device 20 can be implemented by a computing article of manufacture that includes a combination of general and/or specific purpose hardware and/or computer program code.

Similarly, the computer infrastructure 10 is only illustrative of various types of computer infrastructures for implementing the system. For example, the computer infrastructure 10 may include two or more computing devices (e.g., a server cluster) that communicate over a communications link, such as a network, a shared memory, or the like, to perform the processes described herein. Further, while performing the processes described herein, one or more computing devices in the computer infrastructure 10 can communicate with one or more other computing devices external to computer infrastructure 10 using a communications link, including a combination of wired and/or wireless links; a combination of one or more types of networks (e.g., the Internet, a wide area network, a local area network, a virtual private network, etc.); and/or utilize a combination of transmission techniques and protocols.

In one example, the system may be implemented in hardware including:
a computer infrastructure operable to implement a diet identification tool configured to identify qualities of a diet of a user on a graphical user interface based on composite images selectable by the user; and
a composite image tool, wherein the composite image tool is populated with a library of composite images, wherein each composite image depicts a unique inventory of proportions of foods, ingredients, dishes and meals representative of a particular diet quality level X of a particular diet type N for a period of time, and wherein the library of composite images is accessed using the diet identification tool on the graphical user interface, and
at least one of:
1) an environmental sustainability tool to identify an environmental quality of the diet of the user based on composite images selected by the user;
2) a diet optimization tool configured to allow the user to select a different diet type N and/or diet quality level X using the composite image tool and the diet identification tool;
3) a calculation tool configured to calculate personalized nutrient levels and personalized environmental impacts of the user based on inputted information of the user, wherein the graphical user interface is configured to allow the input of the information and to display the calculated personalized nutrient levels to the user;
4) a coaching tool populated with multiple coaching tips includes discrete steps and changes to allow the user to move from one N diet type to a different N diet type and/or from one level of diet quality X to a different level of diet quality X, wherein the graphical user interface is configured to display the coaching tips to the user;
5) a diet tracking tool, wherein the diet tracking tool allows the user to change or update their diet type N, change or update their level of diet quality X, and compare changes in level of diet type N and level of diet quality X over time; and wherein the graphical user interface is configured to display the changes; and
6) a navigation tool populated with discrete steps to move the user stepwise from one level of diet quality X of one N diet type to a different N diet type and/or from one level of diet quality X to a different level of diet quality X, wherein the graphical user interface is configured to display a navigation route to the user.

In one example, the system described herein includes a computer infrastructure operable to implement the diet identification search tool, composite image tool, environmental sustainability tool, diet optimization tool, calculation tool, coaching tool, diet tracking tool, and navigation tool.

In another example, a computer program product includes a computer usable medium having readable program code embodied in the medium, the computer program product including at least one component that when executed by a processor is operable to:
A) display a menu of N diet types for selection by a user on a graphical user interface,
B) display a first group of unique composite images of diet quality levels Xn, wherein each composite image contains images of foods in specific portions and depicts relative portions of ingredients, dishes and meals representative of the level of diet quality Xn of the N diet type selected by the user;
C) display a different group of unique composite images of diet quality levels Xn upon selection by the user of one of the unique composite images in the first group of unique composite images, wherein one composite image of the level of diet quality Xn is the same as in the display of the first group of unique composite images and at least one composite image is different;
D) display an input screen to allow the user to input personal information about the user on the graphical user interface, wherein the personal information may be one or more of gender, age, height, weight, and activity level;
E) display an input screen to allow the user to input diet modification information on the graphical user interface; and
F) calculate a user specific assessment of diet quality and type for the user and display the calculated user specific assessment of diet quality and type to the user on the graphical user interface.

In addition, each unique composite image depicts relative portions of foods, ingredients, and dishes for breakfast, lunch, dinner and snacks over a multi-day period, where the foods ingredients, and dishes exemplify a level of diet quality X of an N diet type.

The diet modification information may include dietary preferences regarding specific ingredients, dishes, meals and/or foods and the input screen allows the user to input additions or subtractions in whole or in part of these specific ingredients, dishes, meals and/or foods. These dietary preferences may include one or more of alcohol, meat, poultry, fish, nuts, water, dairy, vegetables, fruits, refined grains, whole grains, legumes, fast food, sweets, and alcohol. This diet modification information may also include dietary restrictions such as dairy-free, gluten-free, shellfish-free, peanut-free, egg-free, nut-free, wheat-free, soy-free and alcohol-free and the input screen allows the user to input the dietary restrictions

In one example, it is further contemplated that the processes and system may provide a business method that performs the processes described on a subscription, advertising, and/or fee basis. That is, a service provider could offer to perform the processes described. In this case, the service provider can create, maintain, deploy, support, etc., a computer infrastructure that performs the process steps for one or more customers. In return, the service provider can receive payment from the customer(s) under a subscription and/or fee agreement and/or the service provider can receive payment from the sale of advertising content to one or more third parties.

As described herein, the computer program product may contain a coaching tool that is populated with coaching tips including discrete steps and changes to allow the user to modify their diet from one N diet type to a different N diet type and/or from one level of diet quality X to a different level of diet quality X, and in which the graphical user interface displays the coaching tips to the user.

The computer program product may also contain a diet tracking tool. In this instance, the graphical user interface can display an input screen to allow the user to change or update their diet type N, change or update their level of diet quality X, and the graphical user interface is configured to display changes in level of diet type N and level of diet quality X over time.

The computer program product may also contain a navigation tool, wherein graphical user interface displays a navigation route or navigation steps to the user to move the user stepwise from one level from one N diet type to a different N diet type and/or from one level of diet quality X to a different level of diet quality X.

In one example, the techniques for assessing diet type and quality of a user may be implemented using the computer program product described herein, the method including the steps of:
A) selecting an N diet type from the menu of N diets displayed on the graphical user interface, wherein once the user selects the N diet type from the menu of N diets, the graphical user interface displays the first group of unique composite images of diet quality levels Xn, wherein each unique composite image of diet quality level X depicts a different level of diet quality Xn of the selected N diet type;
B) selecting a composite image of diet quality level Xn from the first group of unique composite images, wherein the display instructs the user to select the composite image of diet quality level Xn that approximates the user's current diet, wherein once the user selects the composite image of diet quality level Xn, the graphical user interface displays a different group of unique composite images of diet quality levels Xn,
C) selecting a composite image of diet quality level Xn from the different group of unique composite images of diet quality levels Xn to more closely approximate the user's current diet;
D) optionally, iteratively repeating steps b) and c); In this instance, the step of selecting a composite image from the group of composite images may be repeated multiple times.
E) inputting personal information into the display screen of graphical user interface;
F) inputting diet modification information into the display screen of the graphical user interface, wherein the diet modification information includes dietary restrictions or dietary preferences; and
G) displaying on the graphical user interface a user specific assessment of diet type and diet quality based on the inputted information.

In the step of iteratively repeating steps b) and c); the selection of a composite image may be repeated multiple times. For example, if the user is first presented with images X1 and X2 and selects image X2, the user may then be present with images X2 and X3, If the user selects image X3, the user may then be presented with images X3 and X4. If the user again selects image X3, the selection process may stop, whereas if the user selects image X4, the iterative process may continue until the user is satisfied that they have selected the image that most closely resembles their diet quality level X of diet type N.

This method may further include the steps of:
A) displaying on the graphical user interface a different menu of N diet types, wherein the different menu of N diet types include optimal diets in terms of health and/or environmental sustainability;
B) selecting an N diet type from the different menu of N diet types displayed on the graphical user interface, wherein once the user selects the N diet type from the different menu of N diet types, the graphical user interface displays the first group of unique composite images of diet quality levels Xn, wherein each unique composite image of diet quality level X depicts a different level of diet quality Xn of the selected N diet type;
C) selecting a composite image of diet quality level Xn from the first group of unique composite images, wherein the display instructs the user to select the composite image of diet quality level Xn that approximates the user's goal diet, wherein once the user selects the composite image of diet quality level Xn, the graphical user interface displays the different group of unique composite images of diet quality levels Xn,
D) selecting a composite image of diet quality level Xn from the different group of unique composite images of diet quality levels Xn to more closely approximate the user's goal diet;
E) optionally, iteratively repeating steps b) and c);
F) inputting diet modification information into the display screen of the graphical user interface, wherein the diet modification information includes dietary restrictions or dietary preferences; and
G) displaying on the graphical user interface a user specific assessment of the goal diet type and diet quality based on the inputted information.

According to one example of the system, a Diet Identification Tool 25 may identify a diet of a consumer based on composite images(s) selected by a user on the external I/O device 80. In one example, the external I/O device 80 includes a graphical user interface (GUI). Thus, the consumer accesses computing device 20 through the 1/0 interface 80 and is presented with a series of composite images on the GUI. Upon selection of a composite image and using the system and method described herein, Program Control 50 can then calculate/identify a dietary score for the user based on the composite images selected and display the dietary score to the user on the GUI.

As one example, Composite Image Tool 35 is populated with a library of composite images, wherein the images includes photographs that have been prepared by the methodology described above and include an inventory of foods in specific portions. The composite images each depict ingredients, dishes, and meals included in the particular menu plan, and establish the relative proportions of each variety of food so the quantitative representation is accurate. Composite images contained in the Composite Image Tool 35 can be displayed as images on the GUI of the external I/O device.

In another example, the Environmental Sustainability Tool 45 identifies an environmental quality of a diet type N of a consumer based on the composite image selected by a user on the external 1/0 device 80. Then, the consumer accesses computing device 20 through the I/O device 80 and is presented with a series of composite images on the GUI. Program Control 50 can then calculate/identify an environmental score and display the environmental score to the user on the GUI.

In a further example, Diet Optimization Tool 55 permits a user to identify a different diet type N or level of diet quality X and presents composite images to the user of selection on the external I/O device 80. This different diet may be a different diet type N (i.e., flexitarian versus vegan), different in level of diet quality X and/or different in environmental score. The consumer accesses computing device 20 through the I/O interface 80 and is presented with a series of composite image on the GUI. Upon selection of an image, Program control 50 can calculate/identify a different dietary score or environmental score for the user based on the composite images selected and display the dietary score to the user on the GUI. The diet optimization tool may also include a diet personalization tool in which a user can identify one or more elements of diet to be added, reduced or removed from their diet, including alcohol/wine, meat, poultry, seafood, etc. In one example, the diet personalization tool can allow a user to modify the selected composite image with items that represent their preferences (e.g. swap white wine for red wine in the composite image).

Calculation Tool 65 may be configured to calculate personalized information about the user based on information inputted into the GUI of the I/O device 80. This input may include, for example, personalization information containing the level of diet depicted in the series of images, dietary restrictions, personal information of the user (i.e., gender, age, height, weight, etc.), activity level of the user, and other information. The program control 50 can then calculate personalized nutrient levels of the user based on the composite images selected and information inputted and then display this information to the user on the I/O device 80.

Coaching Tool 75 may be populated with coaching tips including discrete/incremental steps/changes that may be taken by a user to move from an initial level of diet quality X to a different level of diet quality X. Upon selection of a composite image of a desired or optimal diet type N of a user through the GUI of the I/O device 80, program control 50 can access the coaching tips contained in the Coaching Tool 75 and display these tips in a step-wise fashion to the user on the GUI so that the user can incrementally change their diet from a first/baseline diet N and level of diet quality X to a different diet type N and/or level of diet quality X. The coaching tool 75 may be configured to provide substitute or complementary items to the user.

For example, Coaching Tool 75 may be populated with coaching tips such as:

Type of diet: American (highly processed) >>>flexitarian>Mediterranean>>>vegetarian>>>vegan

Meat: Red meat—Grass-fed read meat>>>Red meat once/week>>>Red meat once/month>>>Red meat rarely

Poultry: Chicken/poultry—Free range>>>Organic>>>Substitute one or more meatless meals

Fish: Farm-raised>>>wild caught>>>particular types of fish

Produce: Eat more vegetables/fruits>>>refined/processed>>>canned>>>frozen>>>greenhouse grown>>>organic>>>field grown>>>in season

Shopping; Supermarket>>>farmer's market>>>community sponsored agriculture (CSA)>>>home garden

Packaging; Processed>>>canned>>>plastic-wrapped>>>cardboard>>>minimal>>>none

Origination of goods: International>>>regional>>>local>>>home garden

Level of refinement; heavily processed>>>canned>>>frozen>>>fresh>>>organic

Optionally, the computing device may also include an item tool 85 that is configured to provide a grocery list for the consumer and information about the items on the grocery list for the consumer. The items may be listed on a manufacturer's or merchant's website or on other websites. Additional information such as “green” information, may be obtained about an item. For example, selecting the item may also give descriptive information, for example by a hyper-link to green information on the website or on the world wide web or Internet. The item tool 85 may also be employed to provide a profile which may be used to specify what characteristics of items are to be displayed.

In some instances, a tool may perform one or more functions of another tool, or may employ one or more other tools to perform one or more functions.

### Exemplary Diet Fingerprinting System

Image based systems, methods, and computer-readable media can

dynamically generate image vignettes (i.e., fingerprints) that can be derived from composite images and an image-based diet map representing a spectrum of real-world dietary patterns for a given population, encompassing both baseline diets, and goal diets for diverse health objectives. Like any map, the diet map is populated by coordinates, but rather than latitude and longitude, the diet map coordinates are ‘diet type’ by ‘diet quality’. A unique image, representing a multi-day meal plan, can be formulated for each set of coordinates (i.e., cell in the map) by applying at a minimum two filters: DATs (differentiating attributes by type, that define food requirements, food exclusions, and food allowances for that given diet type) and DAQs (differentiating attributes by quality, defining the dietary elements concordant with the DATs that produce an overall eating patterns achieving a predefined score on the Healthy Eating Index 2015 scale). Each of these multi-day meal plan prototypes have been fully analyzed for nutrient and food group composition—at the “day” level and at the “dish” level. From these entries, the dietary fingerprint is derived as follows.

In order to dynamically generate an image vignette corresponding to fingerprint of a specific composite image, which is a quintessential representation of a multi-day meal plan, the system can dynamically identify the best food dish (or beverage) combination from the total set of possible food dishes (or beverages) in underlying dietary intake meal plans and then dynamically assemble the image vignette as a composition of the best possible food dishes (or beverages).

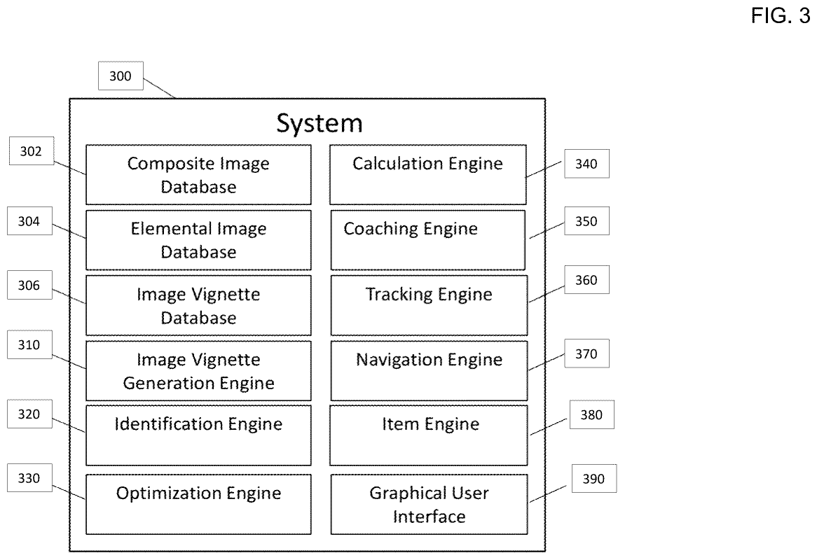

FIG. 3 is a block diagram of an image-based system 300. In an example, the image-based system 300 can be implemented to facilitate dietary assessment and optimization. While an example application of the system 300 can be described with reference to dietary assessment and optimization such a system can also be implemented for other applications. The system 300 can include a composite image database 302, an elemental image database 304, an image vignette database 306, an image vignette generation engine 310, an identification engine 320, an optimization engine 330, a calculation engine 340, a coaching engine 350, a tracking engine 360, a navigation engine 370, an item engine 380, and a graphical user interface 390.

As described herein, composite images have previously been used for an example application of diet quality assessment and optimization to depict a unique inventory of proportions of foods, ingredients, dishes and meals representative of a particular diet quality level X of a particular diet type N for a period of time, which may be one week. The system 300 can provide for the dynamic generation of image vignettes that form digital fingerprints of such composite images such that the visual representation provided by the image vignettes, although reduced, can replace the composite image to reduce the content being displayed and reduce the memory required to store images without significantly detracting from the accuracy and effectiveness of representing the content of the composite image. Users do not need to look at or study composite images of the full dietary pattern because the same, detailed knowledge of a user's dietary pattern can be reached via sequential presentation of the image vignettes, which are much simpler images to process, requiring less processing/computing time and greater case of use by the user. In the same way that a fingerprint represents much less than the full identity of a person but can be mapped to a single, unique identity, small, key features of dietary patterns can be used to map a user's dietary pattern.

To reduce the number of choices available to the user and streamline the process, filters can be used by the system 300 to identify diet preferences and diet restrictions. In one example, other filters could also be used including, for example, where in the world are you and your diet? (region); basic diet character, and whether or not your diet is typical for that region; and so on. Applying just a few, high-level filters can reduce the relevant library for an individual to a much smaller, more easily navigated subset.

The system 300 can create an interface with the composite image library or database 302 that stores or references image files associated with composite images. For an example application of diet quality assessment and optimization, each composite image can be stored in the database 302 as a record and can depict a unique inventory of proportions of foods, ingredients, dishes and meals representative of a particular diet quality level X of a particular diet type N for a period of time (e.g., one week). The composite images can be photographs and can establish relative proportions of each variety of food so the quantitative representation of the food is accurate. The composite image database 302 can be structured as a grid or map where each record corresponding to a composite image is linked with or points to records that constitute the nearest neighbor. The diet types and diet quality levels can form a coordinate system for the map or grid. As one example, a record associated with a first composite image having a specified level X and type N can be linked with or point to records of a composite image having the same type N, but with level X that is incremented and/or decremented by one. As another example, a record associated with a first composite image having a specified level X and type N can be linked with or point to records of a composite image having the same level X, but with a type N that is incremented and/or decremented by one. Employing this grid-based architecture in the composite image database 302, advantageously allows the system 300 to identify not only a record of a specific composite image based on a query that includes a specified level X and a specified type N, but also allows the system to identify those records for additional composite images that are considered the nearest neighbor of the specific composite image. This can allow the system 300 to retrieve additional composite images without executing a query for the additional composite images (e.g., by retrieving images around the composite image in the map or grid). The structure of the composite image database 302 therefore allows the system 300 to not only pin point a specific composite image but to also target an area in the grid or map corresponding to a group of linked composite images associated with a diet type and/or a diet quality level.

To generate the composite image database 302, a validated measure of diet quality can be utilized to identify dietary patterns that each represent a level of diet quality for a period of time and a dietary score can be assigned to each of the dietary patterns. As a non-limiting example, validated measures such as the Healthy Eating Index or the Alternate Healthy Eating Index can be used. For example, the quintiles of the HEI can be used to identify a variety of “real world” dietary patterns representing a level of quality. The dietary score may be assigned to each of dietary patterns taking into account variations in region, culture, diet character and nutritional quality. The dietary patterns can be converted into representative dietary patterns. For example, the diet scores can be mapped back from nutrients, to food and beverage sources, and used to generate representative dietary patterns or “prototypes”. Each such prototype can be readily displayed as “usual” food and beverages consumed, and these, in turn, can be composed into the subject of a composite image. The analytics and specifications for each of the diet types and diet quality levels of the representative patterns can then be calculated and the representative dietary patterns can be converted or translated into the composite images, which can be photographs, where each unique composite image depicts relative portions of foods, ingredients, and dishes for breakfast, lunch, dinner and snacks over a multi-day period, where the foods, ingredients, and dishes exemplify a level of diet quality X of an N diet type. The dietary patterns may include a number of typical dietary patterns for a given population, taking into account “poor,” “good,” “better,” and “best diets for the given population.

The Healthy Eating Index (HEI), as explained, for example, in P. M. Guenther et al., “Update of the Healthy Eating Index: HEI-2010,” Journal of the Academy of Nutrition and Dietetics, Dec. 21, 2012, is a measure of diet quality that assesses conformance to dietary guidelines for Americans. The HEI is routinely expressed in quintiles, i.e., 5 levels of diet quality. For convenience, these might be called: “poor,” “fair,” “acceptable,” “good,” and “excellent.” In addition, there are a variety of specific dietary patterns that could qualify for each quintile based on its composition. Assignment to a given quintile is based on an overall quality score, which is in turn based on nutrient data, which is in turn based on customary food and drink intake reported.

Other validated measures of overall diet quality include the Alternate Healthy Eating Index (AHEI), developed at the Harvard School of Public Health as an ‘alternate’ to the original Healthy Eating Index developed at the USDA. The AHEI is more robustly correlated with health outcomes, including risk of major chronic disease and all-cause mortality.

Other measures of diet quality, including those currently in use or yet to be conceived, can be used to develop the dietary patterns. For example, the dietary patterns can be selected from the group consisting of healthy eating index, alternative healthy eating index, healthy eating index 2010, alternative healthy eating index 2010, diet quality index, healthy eating index from food frequency score, healthy diet indicator, healthy food index, healthy food and nutrient index, recommended food score, diet quality score, diet quality, dietary guidelines index, Mediterranean diet score, Mediterranean adequacy index, alternative Mediterranean diet score, total and specific food group diversity, variations of any of the foregoing and combinations of one of more of the foregoing.

In some instances, the composite images in the composite images database 303 can be developed through an iterative process of ‘tetrangulation’ involving:

Diet Quality Expertise, in which a determination of Principal Differentiating Dietary Components (PDDCs) that differentiate among the quintiles of the HEI-2015 is made for given varieties of diet;

Diet Character Variant Expertise, in which researchers and dietitians with knowledge of real-world results in large epidemiologic studies will help establish parameters for range of variants for a given population;

Expertise in PDDCs, in which expertise in FACTOR ANALYSIS helps link salient dietary factors to differences of both character, and quality; and

Expertise in food choreography, which uses creative oversight of food assemblies suitable for photography, with attention to food placement, emphasis, etc.

Each record in the composite image database 302 can result from input in these four areas, producing an inventory of foods suitable for photography.

To differentiate among diets that are much alike, food placement in the images can emphasize the subtle differences; and/or interactive programming can allow for magnifying subtle differences (e.g., by placing a cursor over an image, it's main differences from a neighboring image are highlighted in text, or by selectively pulling components into the foreground/magnification).

A grid of composite images is shown below in Table 1 as being illustrative of the expandable grid of diet quality.

TABLE 1
Illustrative Grid of Diet Quality

| | | | | |
|---|---|---|---|---|
| SWD-1 | IWD-1 | AWD-1 | GWD-1 | EWD-1 |
| SWD-2 | IWD-2 | AWDv-2 | GWDv-2 | EWDv-2 |
| SWD-3 | IWD-3 | AWDv-3 | GVD-3 | EVD-3 |
| SWD-4 | IWD-4 | AWDv-4 | GAD-4 | EAD-4 |
| SWD-5 | IWD-5 | AWDv-5 | GMD-5 | EMD-5 |

(S = standard; I = improved; A = acceptable; G = good; E = excellent; WD = Western diet; WDv = variant on Western Diet; VD = Vegetarian diet; AD = Asian diet; D = Mediterranean diet)

The system 300 can create and interface with the elemental image library or database 304 that stores or references elemental image files associated with elemental images. For example, each record in the elemental database 304 can correspond to an elemental image. In addition to including or referencing the elemental image file, the record for each element image can include tags or data fields that include data or information related to attributes or characteristics of the content depicted in the elemental image. For an example application of diet quality assessment and optimization, the element images can depict integrated units of food or a beverage and the tags or data fields can include, for example, attributes associated with the integrated units of food or the beverage. An integrated unit of food can be a combination of food that forms a single, distinct dish. For example, an integrated unit of food can be toasted bread or can be a sandwich that includes bread, deli meet and cheese, but not to a meal which typically includes multiple integrated units of foods, such as a beverage, a main dish, and a side dish. The attributes can include defining attributes by type (DATs), such as key dishes, exclusions, inclusions, food group parameters, and/or nutrient parameters that may be used to define a type of dietary pattern; defining attributes by quality (DAQs), such as exclusions, inclusions, food group parameters, and/or nutrient parameters that may be used to define a quality of a dietary pattern; a prevalence defined by population-level data, such as a prevalence of specific dish or beverage categories, which can be derived from published materials and nutritional epidemiology; a frequency with which a dish or beverage appears in a specific diet type; and a proportional contribution indicating contribution of an ingredient, dish, or beverage to an overall diet type by volume or energy.

As a first step in the process for creating the elemental image database 304, food dishes in a multi-day meal plan (representative of a typical prototypical way of eating, defined by a specific diet type and a diet quality level) are identified. A multi-day meal plan can consist of breakfast, lunch, dinner, snacks, and beverages per day. A multi-day meal plan can be generated with fixed entries, or can be dynamically generated with an array or entries. Each food dish has a corresponding visual representation. For example, if one “Breakfast” in a multi-day meal plan is a bagel with cream cheese, the elemental image database can store an isolated image of a bagel with cream cheese which corresponds to an integrated unit of food. This isolated elemental image may be composed of two components, e.g. the bagel as one component and the cream cheese as a second component. The two components can be used to create one single Breakfast image.

Each isolated dish image (elemental image) is tagged with the diet type and diet quality level meal plan from which it originated. For example, if the bagel with cream cheese was found in the multi-day meal plan of the American Style, Quality Level 2 meal plan on day 2, the image would be tagged with “Type=American,” “Quality Level=2,” “Meal=Breakfast,” “Day=2.”

The system 300 can create and interface with the image vignette database 306 that stores or references image vignette files generated by the system 300 based on the composite images of the composite image database 302 and/or based on the elemental images of the elemental image database 304. Each record in the image vignette database 306 can reference or point one of the composite images in the composite image vignette database 302 to map the image vignettes to the grid defined by the composite image database such that each image vignette corresponds to a specific diet level X and data type N for the example application of diet quality assessment and optimization. In some instances, the image vignettes can be stored with their corresponding composite images in the composite image database 302, rather than having a separate image vignette database 306.

The image vignette generation engine 310 can generate image vignettes based on the composite images of the composite image database 302 and/or based on the elemental images of the elemental database 304. In some instances, the image vignette engine 310 can generate at least one image vignette for each composite image of the composite image database 302 or for each possible combination of diet quality level X and diet type N. The image vignettes can be assembled by populating an image vignette file with elements that (a) collectively map to, and exclusively to, a single cell in the diet map (i.e., a particular diet type and diet quality level); (b) capture and convey the general character of that dietary pattern at a glance; (c) allow the end user to choose or reject that ‘dietary fingerprint’ as their own, based on rapid pattern recognition. The image vignette generation process can be standardized by the image vignette generation engine to eliminate the ‘visual burden’ of distraction by movement in dietary components. For example, the image vignette generation engine can define standardized placement locations for discrete dietary components (e.g., beverages are always located in the same location for each image vignette) permits undistracted attention to compositional changes, without changes in placement.

The image vignettes generated by the image vignette generation engine 310 can be stored in the image vignette database 306 for subsequent use and can be linked to records for the composite images in the composite image database 302. In some instances, once the image vignette database 306 is created and include at least one image vignette for each composite image, the system 300 can delete the composite images, while maintaining the grid of the level and type. In some instances, the image vignette engine 310 can generate image vignettes on demand in response to input received from a user. The image vignettes generated on demand can be temporarily stored by the system 300 until they have been exported to a client application being executed on a client device, at which time the system can delete the image vignettes. In systems that utilize on-demand generation of image vignettes, the system 300 may be devoid of the image vignette database 306. Using this approach, the system 300 can actively manage memory allocation to decrease amount of memory required to store images. The effect of this approach can be useful as the number of possible image vignettes increases based on an increase in the number composite images and/or elemental images stored in the databases 302 and/or 304, respectively, because the system 300 does not have to allocate dedicated memory resources to the image vignettes.

The image vignettes can form “dietary fingerprints” which are a composed assembly of food images used to identify the particular diet quality level and diet type of the user in an efficient manner. The compositions captured in the image vignettes can characterized as “fingerprints” because they function as human fingerprints do: a very tiny portion of the whole can be mapped reliably, and exclusively, to the one “whole” it represents. In the case of human fingerprints, that is a person; in the case of dietary fingerprints, that is a specific, operationally defined type of dietary intake pattern at a specific, objectively quantified tier of overall nutritional quality. In some instances, each of these unique image vignettes can be derived from a corresponding unique composite image and/or a 3-day meal plan for that corresponding “cell” in the diet map or grid (a prototype of a distinct diet type and quality level). For example, the image vignettes can be visual representations of a prototypical 3-day meal plan for that particular diet quality level and diet type.

In this regard, each image vignette can represent signature food characteristics of the particular diet quality level X of diet type N, which may include a small number of foods that most accurately reflect both the particular diet quality level X and particular diet type N. In one instance, this small number of foods may include approximately three to about ten foods, or four to eight foods, but may be another number that can accurately reflect the characteristics in a simple and straightforward manner. For example, a “worst case” low diet quality level of an image vignette of an “American” diet may include images of soda, pizza, French fries, bacon, cookies/desserts and be devoid of vegetables. On the other hand, a “high quality” high diet quality level of an image vignette of a Mediterranean diet may include images of steel cut oats, salad, fish, fruits/vegetables, non-fat yoghurt and water.

In one implementation of the system 300, the image vignette generation engine 310 can generate the image vignettes from the composite images. The image vignette generation engine can query the database of composite images and retrieve an image file that corresponds to a specific composite image from memory based on the tags in the database. As an example, the query can include the coordinates of the specific composite image in the grid having a specified diet type and diet quality. The image vignette generation engine 310 can perform one or more imaging processing and/or machine vision to process the composite images and extract elements from the composite images. For example, the image vignette generation engine 310 can use Stitching/Registration, Filtering, Thresholding, Pixel counting, Segmentation, Inpainting, Edge detection, Color Analysis, Blob discovery and manipulation, Neural net processing, Pattern recognition, Optical character recognition, blurring, normalized lighting, greyscaling, OTSU, thresholding, erosion/dilation, convert correct hull, contour detection, blob/mass calculation normalization, and/or Gauging/Metrology to extract elements from the composite image based on, for example, a hierarchical algorithm. The image vignette generation engine 310 can create a new image file corresponding to an image vignette to be generated based on the extracted elements, where the new image file defines an image canvas. The image vignette generation engine 310 can insert the extracted elements from the image file for the composite image into the image canvas of the new image file at specified locations.

In one example of the system 300, the image vignette generation engine 310 can generate the image vignettes from the elemental images. The image vignette generation engine can query the elemental images database to retrieve a set image files from memory corresponding to elemental images based on the tags in the elemental image database. The system 300 may generate one or more queries for elemental images having tags that correspond to one or more specified dietary patterns. The image vignette generation engine 310 can select a subset of the image files based on a hierarchical algorithm described herein that receives as an input the tags associated with the elemental images. As an example, the one or more processors can execute the hierarchical algorithm to iteratively eliminate image files from the set of retrieved image files based on the tags associated with the elemental images associated with the image files. Once the subset of elemental images is created, the image vignette generation engine 310 can extract the elemental images from the image files associated with the subset of the selected elemental images insert the extracted elemental images from the image files of the subset into an image canvas of a new image file created by the image vignette generation engine 310. As described herein the elemental image can be inserted into the image canvas at specified locations.

For example, each multi-day meal plan (e.g. Y-day meal plan) for each cell in the diet map for the composite images may consist of the following:
Y breakfasts
Y lunches
Y dinners
Y or more beverages
Y or more snacks

Each dietary fingerprint represented by an image vignette incorporates a set of Z number images arranged into a standardized architecture, and can consist of at least one of the following items, one in each slot in the image vignette.
Breakfast
Lunch
Dinner
Beverage
Snack

In order to dynamically identify the best Z number of dish (or beverage) images (elemental images) from the total array of images from the Y-day meal plan, the image vignette generation engine 310 can utilize hierarchical series of inter-dependent filtering algorithms illustrated by the below table.

| | |
|---|---|
| DATs (Defining attributes by type, | Entry must be compatible |
| e.g. key dishes, exclusions, | |
| inclusions, food group parameters, | |
| and/or nutrient parameters) | |
| DAQs (Defining attributes by | Entry must be compatible |
| quality, e.g. exclusions, inclusions, | |
| food group parameters, | |
| and/or nutrient parameters) | |
| Prevalence (Defined by | Prioritize entries eaten by |
| population-level data about | ‘nearly everyone’ in |
| prevalence of specific dish | given population |
| categories, derived from published | |
| materials and nutritional | |
| epidemiology) | |
| Frequency (Defined as a frequently | Prioritize entries eaten most |
| occurring dish, beverage, or “entry” | often in the DietType and/or |
| in our cells.) | the Diet Quality Level |
| Proportional contribution | Prioritize entries making largest |
| | proportional contribution to overall |
| | diet type by volume or energy |
| Representativeness | Prioritize entries most strongly |
| | associated with diet type (e.g., |
| | foods most ‘integral’ to DATs) |
| Exclusivity | Prioritize entries that best |
| | differentiate between a given diet |
| | and its neighbors in the diet map |
| Redundancy | Establish upper bounds for entry |
| | recurrence. IF already display X |
| | times, THEN suppress at X + 1 IF |
| | there is a suitable alternative, ELSE |
| | IF there is no suitable alternative, |
| | then display X + 1st time. |
| Reciprocals/Alternativality | If entries A and B work for a given |
| | cell in sequence, but only B works |
| | for higher quality cells, then |
| | prioritize A as initial selection. |

Using the above algorithm, the image vignette generation engine 310 can select a specified number of elemental images for a specified number of slots in the image vignette to be created. For example, the image vignette generation engine 310 may select eight food dishes represented by the elemental images that can be used to form the image vignette (e.g. one breakfast dish, two snacks, two dinner dishes, two lunch dishes, and a beverage). The image vignette generation engine 310 can form the image vignettes so that each category of dish (e.g., breakfast, lunch, dinner, snack, beverage) are always located in the same area of the image vignettes so that slight changes from one image vignette to another image vignette can be readily perceived by a user. To achieve this, the image generation engine 310 can resize the content extract from the elemental images to fit within the designated area so that the scale between content represented by each elemental image is maintained.

The system 300 can be programmed and/or configured to adapt the image vignettes to the display device upon which the image vignettes are to be rendered based on a property of the client device In this regard, the system 300 can control the process for generating and rendering the image vignettes in a graphical user interface of a client application on a client device. As an example, a number of image vignettes generated and exported by the system 300 for each iteration can be dependent on one or more properties of the client device, such as an available memory capacity, a network connection speed, a type of display technology, a size of screen of the display, a resolution of the display, and the like. The system 300 can determine the one or more properties of the client device based on communication with the client device. More image vignettes can be generated and/or exported for larger screens or device with more available memory and fewer image vignettes can be generated and/or exported client device with smaller screens or less available memory. As another example, both the size and the number of images visible in the screen can be dynamically adjusted by the image vignette generation engine. On a larger screen, the image vignettes can be displayed side by side, while on a smaller screen (such as a smart phone screen), the image vignettes can be displayed vertically (one above the other) if the smaller screen is detected to be held in a portrait viewing orientation or can be displayed side-by-side if the smaller screen is detected to be held in the landscape viewing orientation. For even smaller screens, such as on a smart watch, the system 300 alternate displaying among the image vignettes. In addition, depending on the size of the screen, certain functions, such as the ability to navigate to composite image corresponding to an image vignette and/or the ability to hover over an image vignette to initiate a magnifying glass function to zoom in on a portion of the image vignette can be disabled. While the number of derivative image vignettes displayed may be any number greater than 1, in most implementations the number of images displayed is between 2 and 50 images, more preferably between 3 and 20 images, and even more preferably between 4 and 10 images. In one example, the number of derivative image vignettes displayed on the graphical user interface is 2.

The identification engine 320 can be programmed and/or configured to present image vignettes to a user by rendering the image vignettes in a graphical user interface on a client device being operated by the user. Initially, the identification engine 320 can generate and/or select one or more sets of image vignettes (associate with different levels and/or types) to display to the user, where the user can select image vignettes from the sets and the identification engine 320 can use the selections converge on one of image vignettes as a starting point of the user (e.g., a starting level and type). For an example application of diet quality assessment and optimization, the identification engine 320 can determine, based on the user's selection of one or more image vignettes via the graphical user interface, the user's current/baseline diet quality level X and current/baseline diet type N. The identification engine 320 can calculate/identify a dietary score for the user based on the selected image vignettes, which can be rendered in the graphical user interface to display the dietary score to the user. The closest approximation to the subject's dietary pattern from the entire library of composite images (best fit) can be identified based on the selection of the image vignettes. This dietary pattern corresponds to specific, well-known nutrient intake levels/1000 kcal.

In one example, the identification engine 320 can render a sequence of image vignettes that correspond to the four corners of the grid or map in the graphical user interface to depict extremes in “best” and “worst” types and levels of diet quality.

The optimization engine 330 can be programmed and/or configured to present image vignettes to a user by rendering the image vignettes in a graphical user interface on a client device being operated by the user. The optimization engine 330 can be executed after a starting point has been identified by the identification engine 320. The optimization engine 330 can generate and/or select one or more sets of image vignettes (associate with different levels and/or types) to display to the user, where the user can select image vignettes from the sets and the identification engine 320 can use the selections converge on one of image vignettes as an end point of the user (e.g., an end level and type). For the example application of diet quality assessment and optimization, the optimization engine 330 can determine, based on the user's selection of one or more image vignettes via the graphical user interface, the user's end/goal diet quality level X and end/goal diet type N. The optimization engine 330 can also comprise a personalization module 332 through which a user can identify one or more elements of diet to be added, reduced or removed from their diet, including alcohol/wine, meat, poultry, seafood, etc.

In one example application of diet quality assessment and optimization, the calculation engine 340 can be configured to calculate personalized nutrient levels and personalized environmental impacts of the user based on information input by the user via the graphical user interface, wherein the graphical user interface includes data entry fields to receive the input of the information from the user and data output fields to display the calculated personalized nutrient levels to the user. The input may include, for example, personalization information containing the level of diet depicted in the series of images, dietary restrictions, personal information of the user (i.e., gender, age, height, weight, etc.), activity level of the user, and other information, and can obtain additional guidance, related, for instance, to recommended calorie intake; serving sizes; etc.

The calculation engine 340 can then calculate personalized nutrient levels of the user based on the image vignettes selected and the input information and then display this information to the user via the graphical user interface. The calculation engine 340 can calculate a user specific assessment of diet quality and type for the user based on the selected image vignettes and can display the calculated user specific assessment of diet quality and type to the user in the graphical user interface.

The calculation engine 340 can also quantify the user's nutrient intake to establish a habitual calorie level. For example, the user's nutrient intake to establish a habitual calorie level calculated with height, weight, sex, age, and activity level, using metrics such as the Harris-Benedict or Mifflin-St. Jeor equations (and/or other suitable methods) for determining basal metabolic rate. Using this approach, the calculation engine 340 estimates total calorie requirements for a user, and can adjust the dietary parameters from the assessment to right-size” the diet for the user such that the diet is personalized for the user. In some instances, information related to energy expenditure of a user (e.g., from physical activity) can be captured from one or more devices (e.g., wearable device, such as smart watches, heart rate monitors, fitness trackers, etc.) worn by the user. These connected devices can be connected (either wirelessly or wired) to the client device of the user and the information collected by these devices can be used by the calculation engine 340 when establishing personalized nutrient parameters and/or caloric parameters for the user. The system 300 may determine a specific diet type of the user; determine a specific diet quality of the user; and can customize or personalize (right-size) the dietary assessment as well as the steps required by the user to optimize their diet to achieve a goal diet type and quality level.

The coaching engine 350 can be populated with coaching tips corresponding to discrete steps and changes a user can follow to move from one type N to a different type N and/or from one level of quality X to a different level of quality X. The coaching engine 350 can selectively render the coaching tips in the graphical user interface to display the coaching tips to the user. In one example application of diet quality assessment and optimization, the coaching engine 350 can selective provide coaching tips corresponding to discrete steps and changes to allow the user to modify their diet from one diet type N to a different diet type N and/or from one level of diet quality X to a different level of diet quality X. In some instances, the coaching tips can provide discrete/incremental steps/changes that may be taken by a user to move from an initial level of diet quality X to a different level of diet quality X. Upon converging on an image vignette to establish a starting point, the coaching engine 350 can render one or more coaching tips in the graphical user interface to display the one or more coaching tips to the user, where the coaching tips describe a step-wise fashion for incrementally changing the diet from a first/baseline diet N and level of diet quality X to a different diet type N and/or level of diet quality X. The coaching tool 350 may be configured to provide substitute or complementary items to the user. Some examples of coaching tips include:

Type of diet: American (highly processed)→flexitarian→Mediterranean→vegetarian→vegan

Meat: Red meat→Grass-fed read meat→Red meat once/week→Red meat once/month→Red meat rarely

Poultry: Chicken/poultry→Free range→Organic→Substitute one or more meatless meals

Fish: Farm-raised→wild caught→particular types of fish

Produce: Eat more vegetables/fruits→refined/processed→canned→frozen→greenhouse grown→organic→field grown→in season

Shopping: Supermarket→farmer's market→community sponsored agriculture (CSA)→home garden

Packaging: Processed→canned→plastic-wrapped→cardboard→minimal→none

Origination of goods: International→regional→local→home garden

Level of refinement: heavily processed→canned→frozen→fresh organic

The tracking engine 360 can provide a graphical user interface that allows the user to change, update, or view the type N and the level of quality X currently assigned to the user; change or update the type N and the level of quality X corresponding to the end point; and/or compare changes in type N and level of quality X assigned to the user over time. For example, the graphical user interface can display an input screen to allow the user to change, update, or view their diet type N; change, update, or view their level of diet quality X, and can display changes in level of diet type N and level of diet quality X assigned to the user over time.

The navigation engine 370 can generate discrete steps to move the user stepwise from one level of quality X of one type N to a different type N and/or from one level of quality X to a different level of quality X. The navigation engine 370 can render a navigation route in the graphical user interface showing the user how to advance from the starting point/baseline to the end/goal point. The navigation route can be rendered as a sequence of the image vignettes corresponding the levels of qualities X and/or types N between the starting point and the end point. For example, the navigation route or navigation steps rendered in the graphical user interface by the navigation engine 370 can provide a stepwise route the user can follow from one diet type N to a different diet type N and/or from level of diet quality X to a different level of diet quality X.

In one example application of diet quality assessment and optimization, the item engine 380 can be configured to provide a grocery list for a user and information about the items on the grocery list for the user. The items may be listed on a manufacturer's or merchant's website or on other websites. Additional information, such as “green” information, may be obtained about an item. For example, selecting the item may also give descriptive information, for example by a hyper-link to green information on the website or on the world wide web or Internet. The item engine 380 may also be employed to provide a profile which may be used to specify what characteristics of items are to be displayed.

The graphical user interface 390 can be configured to provide one or more graphical user interfaces (GUIs) through which users of the system 300 can interact with the system 300. The GUIs can be rendered on display devices and can include data output areas to display information to the users as well as data entry areas to receive information from the users. For example, data output areas of the GUIs can output image vignettes as well as information associated with a user, such as diet score, nutrient and calorie calculations, navigation paths/routes, item lists, and the like, and the data entry areas of the GUIs can receive, for example, information associated with the users. Some examples of data output areas can include, but are not limited to text, graphics (e.g., graphs, maps (geographic or otherwise), images, and the like), and/or other suitable data output areas. Some examples of data entry fields can include, but are not limited to text boxes, check boxes, buttons, dropdown menus, scroll bars, hyperlinks or other selectable links, which may be embedded for example in one or more image vignettes, and/or other suitable data entry fields.

In some instances, the graphical user interface 390 can provide an input screen to allow the user to input personal information about the user in the graphical user interface 390. For example, the graphical user interface 390 can allow the user enter information regarding dietary restrictions or dietary preferences prior to or after being presented with the display of the image vignettes or at another time there between. In some instances, at least some of the information can be automatically obtained such that the user is not required to manually enter the information. As one example, the system 300 can receive geographic location information from a location enabled client device (e.g., a smart phone or other GPS enabled device) of the user based on GPS coordinates of the client device and/or can estimate the geographic location of the user based on an Internet Protocol (IP) address through which the client device is communicating. The geographic location can be used to narrow reduce the relevant library of image vignettes to start from for an individual to a much smaller, more easily navigated subset. As another example, information related to a user's physical parameters and energy expenditure can be collected for user by the system 300 through one or more connected devices (e.g., wearable device, such as smart watches, heartrate monitors, fitness trackers, etc.) which can be connected to the client device (either wirelessly or wired). Such information can be used by the system 300 when personalizing “right-sizing” a user's diet. Using the information input by the user or automatically provided via the client device or associated connected devices, the graphical user interface can be populated with a first set of image vignettes for selection by a user on a graphical user interface that are appropriate for the user based on their dietary restrictions and/or dietary preferences.

The input screen can allow the user to input diet modification information, such as dietary preferences regarding specific ingredients, dishes, meals and/or foods and the input screen can allow the user to input additions or subtractions in whole or in part of these specific ingredients, dishes, meals and/or foods. These dietary preferences may include one or more of alcohol, meat, poultry, fish, nuts, water, dairy, vegetables, fruits, refined grains, whole grains, legumes, fast food, sweets, and alcohol. This diet modification information may also comprise dietary restrictions such as dairy-free, gluten-free, shellfish-free, peanut-free, egg-free, nut-free, wheat-free, soy-free and alcohol-free and the input screen allows the user to input the dietary restrictions.

In some instances, the graphical user interface 390 can present the user with one or more queries to answer some baseline or intake questions so that the first set of images vignettes displayed is tailored to dietary preferences of a user. For example, these queries may analyze whether a user cats meat or cats grain products. Thus, in the case of a user indicating that they are vegetarian or vegan, the first set of derivative images would be plant-based, and the user would not be presented with meat-based derivative image vignettes during the selection process. Thus, a few simple on-boarding questions (i.e., “do you eat meat?”; “where in the world are you” (region); basic diet character; and whether or not your diet is typical for that region; and so on.) are processed by the system 300 to determine which image vignettes to initially display in the graphical user interface 390 to the user.

In one example operation, the system 300 renders a first set of unique image vignettes of diet quality levels Xn, in the graphical user interface for selection by a user. Upon selection by the user of one of the unique image vignettes in the first set of unique image vignettes, the system 300 can render a different plurality of unique derivative image vignettes of diet quality levels Xn in the graphical user interface for selection by the user. For example, if the user is first presented with images X1 and X2 and selects image X2, the user may then be present with images X2 and X3. If the user selects image X3, the user may then be presented with images X3 and X4. If the user again selects image X3, the selection process may stop, whereas if the user selects image X4, the iterative process may continue until the processor determines that the selected derivative image vignette most closely resembles the user's current diet quality level X of diet type N. The system 300 can use this image vignette selection process to determine a current diet type and a current diet quality of the user and/or can use this image vignette selection process to determine a goal diet type and a goal diet quality level for the user. Once the system 300 determines the diet type and diet quality of the user, the system can further personalize the diet quality assessment of the user and/or personalize a diet plan for the user to help the user reach the user's goal diet type and quality level. For example, the system 300 can “right size” the users diet based on personal information provided by the user and the determined current and/or goal diet type and the current and/or goal diet quality using for example, metrics such as the Harris-Benedict or Mifflin-St. Jeor equations (and/or other suitable methods)

In one example operation, once the image vignettes are generated by the system 300 as described herein, a sequence of image vignettes can be rendered in a graphical user interface 390 on the display of a client device. The graphical user interface 390 can include a first scroll bar associated with a first set of derivate image vignettes rendered in the graphical user interface for selection by the user. Each image vignette in the first set can depict dietary characteristics of a particular diet quality level X of a particular diet type N. The first set of image vignettes associated with the first scroll bar can comprise image vignettes depicting a range of quality levels X of a range of diet types, e.g., XaNb to Xa+nNb+m, wherein Xa represents a lowest diet quality level of the range and Nb represents a lowest quality diet type rendered in the first set, and wherein Xa+n represent an improvement in diet quality that is “n” degrees away from the diet type represented by Xa in the grid and Nb+m represents a diet type that is “m” degrees away from the diet type represented by Nb in the grid. The values of n and m can each represent a number greater than or equal to one. Upon selection of one of the image vignettes associated with the first scroll bar by the user, a secondary scroll bar can be rendered in a graphical user interface on the display of the client device that is associated with a second set of image vignettes for selection by the user. At least one of the image vignettes in the second set of image vignettes can be different from the image vignettes in the first set image vignettes. In response to selection of one of the image vignettes in the second set of image vignettes associated the secondary scroll bar by the user, the system 300 executed by the one or more processors determines the user's specific type of diet. Once the system 300 determines the diet type, in response to selection of one of the image vignettes in the second set, a third set of image vignettes can be rendered in the graphical user interface and can be associated with a tertiary scroll bar. At least one of the image vignettes in the third set of image vignettes can be different from the image vignettes in the second set of image vignettes. In response to selection of one of the image vignettes in the third set of image vignettes associated with the tertiary scroll bar by the user can be used by the processor executing the system 300 to determine the user's specific level of diet quality. The graphical user interface rendered on the client device can allow a user to input personal information about the user and the system can calculate a user specific assessment of diet quality and type and estimate of caloric intake and nutrient data for the user. The calculated user specific assessment of diet quality and type and estimate of caloric intake and nutrient data can be rendered in the graphical user interface.

In some instances, the first scroll bar includes image vignettes that are representative of the most extreme “worst” diet quality levels of particular diet types and the most extreme “best” diet quality levels of particular diet types and other varying diet quality levels of particular diet types. By being presented with extremely different types of diets and levels of diet quality, this first scroll bar enables the user to make an initial selection of a type of diet and level of diet quality that reflects the character of their diet. Secondary scroll bars and tertiary scroll bars can then be used to refine this initial selection to hone in on a closer approximation of the type of diet and level of diet quality of the user and the user may cycle through these secondary and tertiary scroll bars multiple times if needed to reach the “best fit” of an image vignette that most closely approximates their diet type/level or diet quality. It is also noted that the user only cycles through the derivative image vignettes displayed on the graphical user interface but is not “guessing” their own level of diet quality or type of diet. It is the selection of the image vignettes by the user that allows the processor executing the system 300 to determine the user's type of diet and level of diet quality based on the images selected.

This information can then be personalized to the user by providing an input screen in which the user can input personal information regarding the user and this personal information personal information may comprise one or more of gender, age, height, weight, and activity level.

In some instances, the system 300 can be programmed and configured to render the image vignettes in the graphical user interface as a fingerprint corresponding to the larger composite image for the particular diet type and diet quality level. The user can access a particular composite image associated with a particular derivative image vignette (e.g., by selecting an option in the graphical user interface 390 to view the corresponding composite image). In some instances, the function that allows the user to view a composite image corresponding to an associated image vignette can be disabled by the system if the system 300 determines that a size of the display upon which the graphical user interface 390 is being rendered does not satisfy a threshold size and can be enabled when the system 300 determines that the size of the display does satisfy the threshold size. In some instances, the function that allows the user to use a magnifying glass function to hover over a portion of an image vignette to magnify that portion can be disabled by the system if the system 300 determines that a size of the display upon which the graphical user interface 390 is being rendered does not satisfy a threshold size and can be enabled when the system 300 determines that the size of the display does satisfy the threshold size. As an example, the system 300 can disable the function when the graphical user interface is rendered on a display of a mobile phone and can enable the function when the graphical user interface is rendered on a display of a laptop or personal computer.

In some instances, the system 300 can be linked to a fitness interface, so that a user is receiving guidance for diet and physical activity improvements concomitantly. While this is optional, in this mode, the diet tips provided can be adjusted to address the physical activity pattern and goals. Furthermore, an interactive system can be used to “plug in” to the system 300 described herein to provide access to recipes/options.

It is specifically contemplated that user selections and/or the results gleaned may be in communication with third party servers or applications. Further, an interactive system may further include a step counter/pedometer. When a user walks or jogs, the system may inform the system of the total amount of calories that a user has expended, and the interactive system may inform the amount of calories a user has consumed or the quality of the diet consumed. It is contemplated that the system may compare expended and consumed calories. A remote server and communication means may connect the above systems to each other. The system may further include storage or memory for storing nutrition information, nutrition/energy taken, the nutrition/energy expended. A user may input the nutrition facts/energy into the storage through a keypad or other methods.

Because dietary intake assessment with the process described herein is almost instantaneous, nearly effortless, and potentially even fun, the process described herein allows for limitless applications in apps, interactive websites, and games. Identification of a “goal” diet is as streamlined as identification of baseline diet and with attention to the incremental dietary changes along the way from baseline to goal, the process described herein is designed to identify key, desirable dietary changes; to address these changes in a logical sequence; and to “coach” the process of dietary change. The platform can function in this manner on its own (i.e., app, website, wearable health tech) or can be used by to enhance the guidance of a human health coach.

As will be appreciated by one skilled in the art, the system may take the form of entirely hardware, entirely software (including firmware, resident software, micro-code, etc.) or a combination of software and hardware that may all generally be referred to herein as a “circuit,” “module” or “system.” Furthermore, the system may take the form of a computer program product embodied in any tangible medium of expression having computer-usable program code embodied in the medium.

The system 300 can include computer program code for carrying out operations and may be written in a combination of one or more programming languages, including an object-oriented programming language such as JAVA, SMALLTALK, C++ or the like and conventional procedural programming languages, such as “C” or similar programming languages as well as one or more scripting languages, such as Python, JavaScript, Rails, Ruby, and the like. The program code may execute entirely on the user's computer, partly on the user's computer, as a stand-alone software package, partly on the user's computer and partly on a remote computer or entirely on the remote computer or server. The remote computer may be connected to the user's computer through different types of networks, including, for example, a local area network (LAN) or a wide area network (WAN), or the connection may be made to an external computer (for example, through the Internet using an Internet Service Provider).

### Determining Dietary Impacts on Environmental Measures

Among the salient influences on planetary health, dietary patterns practiced at scale by Homo sapiens deserve special attention for a number of reasons. First, overall diet quality does not merely influence human health, but stands out as the single leading predictor of risk for both premature death from all causes and incident chronic disease in the modern world. Second, and fortuitously, dietary patterns associated with optimal human health correspond all but perfectly with dietary patterns associated with optimal planetary health (see, for instance: Clark M A. Springmann M. Hill J. Tilman D. Multiple health and environmental impacts of foods. Proc Natl Acad Sci U S A. 2019 Nov. 12;116(46):23357-23362). Third, patterns of food production, driven ultimately by patterns of food demand, impact directly nearly every important metric of planetary health and sustainability: water utilization; land use; greenhouse gas emissions; eutrophication/soil degradation; and more. Fourth, profit incentives associated with prevailing food preferences that discount environmental impacts entirely, invite the degradation of uniquely valuable and iconic ecosystems into yet more pasture and/or arable land. Fifth, the net effects listed above, and others omitted, translate into a systematic transformation of biomass on land from its native diversity into a colossal population of cows, pigs, and chickens with a comparable consolidation of biomass now underway for marine species due to commercial fishing and aquaculture. And finally, shifts in dietary pattern are among the few important impacts on climate and planetary health actionable, at least in high-income nations, at the individual level.

As described above systems providing diet quality photo navigation enable interactions between an individual user's computing device and a network-accessible computer system in order to determine composition of dietary intake and provide an objective measure of overall diet quality for that user. The diet quality measure may be an adaptation of the most current version of the Healthy Eating Index (HEI; such as the Healthy Eating Index 2020 or a later version), a robustly validated, widely used metric in the public domain. In embodiments of the present invention, the different individual dietary elements (i.e. food or beverages) making up the determined dietary pattern are also programmatically mapped to databases of quantified environmental impact across four domains: land use; water use; eutrophication/nitrogen use; and greenhouse gas emissions. For each dietary element contributing to a given diet, these four component measures, which are adjusted in the case of water for the intensity of use varied by site of food production, are aggregated by the system into an overall ‘environmental impact score’ for the dietary element(s) in question. In the case of water use, databases may quantify water utilization based on the region of food production, and the intensity of demand on available fresh water by location. For example, in some embodiments, where water is in high demand and in short supply, such as the Central Valley in California, the impact score of food production in the water utilization category may be amplified by a high weighting coefficient. Where water is more plentifully available relative to demand, such as sites in Florida, the same foods may receive a lower water utilization component score because of a lower weighting coefficient representing the site-specific intensity of water demand related to the production of a given food. The other component measures are less site-sensitive, and are thus may be standardized across sites without weighting coefficients. These cumulative environmental impact scores for dietary elements are further aggregated into a summary score for the entire diet (baseline, and goal) identified by the system for a given user. This is done by establishing the proportional contribution of each dietary element, by calories/energy, to the diet identified.

The cumulative score for the total dietary impact on environmental measures (DIEM score) is situated relative to the full range of scores achievable across the expanse of the diet map. A ratio is established for the DIEM score of a given diet to the maximal score across all diets, and these ratios are stratified by computer algorithm, and converted to a numerical scale (e.g., 1-10) in which, as one non-limiting example, the highest number represents the ‘best’ diet for the environment, and the lowest number the ‘worst.’ These scores are displayed to an end user via computer application that showcases, in the case of goal diets, multiple ‘suitable’ diets (with regard to health objectives) with differing DIEM scores, and invites the end user to select an option that both addresses their personal health goals, and provides the ‘best’ environmental score. The scoring range may be continuous across all diet types and qualities.

Embodiments of the present invention extend the ability to assess diet quality for individual users to the realm of planetary health by considering the effect on the environment of larger groups following and/or adopting certain diets. This is relevant globally, at a massive scale. Embodiments offer the potential to leverage even small shifts in dietary patterns at population scale to help advance planetary health/climate/environmental/sustainability objectives.

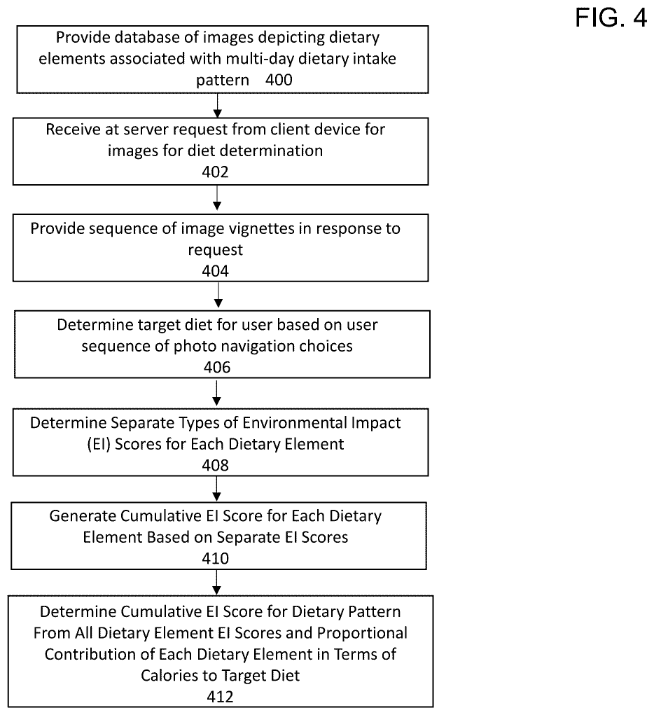

FIG. 4 depicts an exemplary sequence of steps followed by an embodiment of the present invention to determine an environmental impact of a user's target diet, i.e. the user's current baseline diet, one or more goal diets or both baseline and goal diets. The sequence begins with the identification of the user's current dietary pattern. To start this process one or more databases of images that depict dietary elements (food/beverages) representative of a multi-day dietary intake pattern are provided (step 400). In response to a request from the user's client device for images to be used in identifying the user's diet (step 402, a sequence of image vignettes are generated from the database of images and provided (step 404). Selections are made until a “best fit” is achieved, representing the user's current, or baseline dietary pattern. Both a diet quality and environmental impact (DIEM) score are then generated instantaneously. The same method involving user selection between or among displayed images of dietary patterns allows for the identification of a personalized goal diet. As discussed above, a target diet that includes a diet type and quality is determined for the user based on the sequence of photo navigation choices made by the user from among the provided sequence of image vignettes (step 406) In one embodiment, the target diet that is determined for the user is further customized based upon additional factors, including the user's age, weight, sex, height and activity level with the activity level determined in part using data collected from a wearable device tracking the user's activity level that is communicatively coupled to the client device. Once the diet type and quality have been established, the individual dietary elements in that determined dietary pattern are mapped to databases accessible by the integrated system of hardware and software described herein to access the separate scores of environmental impacts in at least four areas (step 408). More specifically, one or more environmental impact databases hold previously determined EI scores leading to the determination of component dietary element scores such as, but not limited to, land use EI scores, water use EI scores, Nitrogen use/eutrophication EI scores, and greenhouse gas emission EI scores, for each dietary element. The four scores are aggregated to determine a cumulative EI score for the particular dietary element (step 410). The sequence then determines whether there are more dietary elements in the dietary pattern that have not yet been determined. If there are additional dietary elements in the dietary pattern, the sequence iterates until all of the dietary elements have been assessed. When there are no more unassessed dietary elements remaining, the sequence determines a cumulative EI score for the user's determined overall dietary pattern based on a weighted aggregate of the individual dietary element compound scores (step 412). As noted above, the determination of the cumulative EI score for the entire dietary pattern involves the weighting of the individual dietary element scores based on their contribution to the diet. For example, for a given dietary pattern, the proportion of each food based on caloric contribution to the dietary pattern is retrieved from the databases and examined and the dietary pattern score is adjusted based on the relative percentages of total calories allocated to given sources. As a result, a dietary element that appears only infrequently in the diet, and thus provides a lower percentage of the overall caloric count, will see its influence on the final DIEM score downgraded while a core element that contributes more calories will see its influence on the final DIEM score increased to reflect its more frequent appearance in the diet.

In some embodiments, after aggregating the four component measures, assigning weights to each food based on the percentage of total daily calories it represents on average and then aggregating the food-level scores to generate an EI score for whole diet, embodiments relatively rank all diets based on the determined scores. In some embodiments, guidance is provided to a user showing which diets in a group of diets have a higher EI to a lower EI (or vice-versa). In further embodiments, such EI guidance may be combined with a user's personal health goals. For example, in one embodiment, the system described herein may recommend a dietary pattern, from among multiple diets, based on a user's personal health goals that has the lowest EI and/or recommend a dietary pattern to be chosen from among a group of dietary patterns with low EIs based on the user's personal health goal.

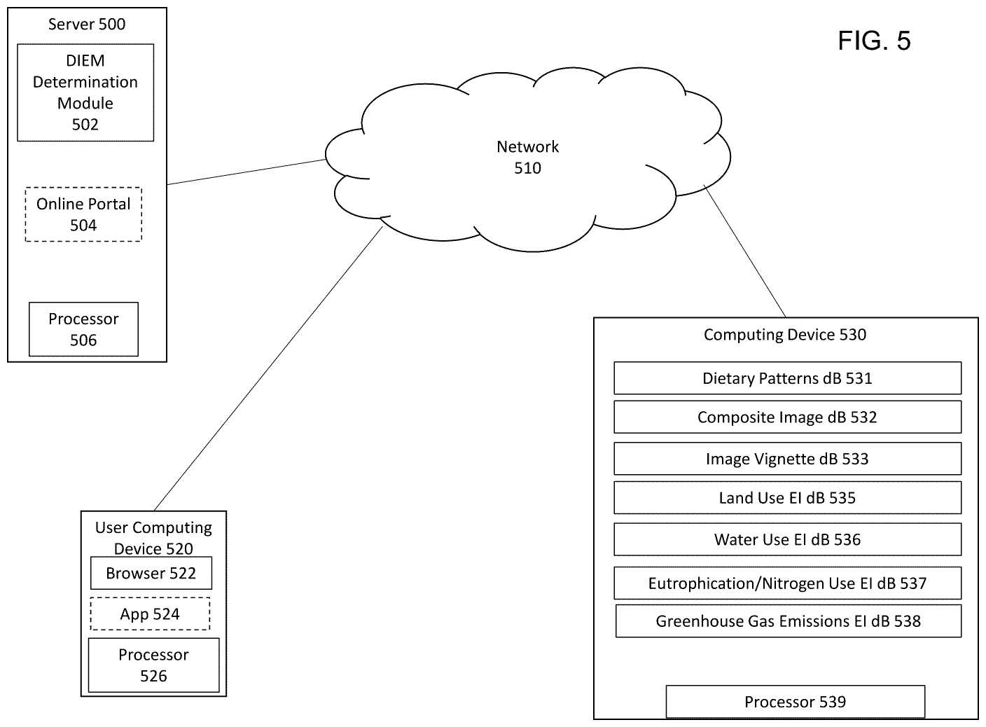

FIG. 5 depicts an exemplary environment suitable for practicing one or more embodiments of the present invention. The exemplary environment, includes one or more servers 500 that are configured to execute a DIEM determination module 502 with the aid of one or more processors 506. The DIEM determination module 502 may be implemented as one or more software applications configured to perform some or all of the DIEM determination sequence discussed in FIG. 4, alone or in parallel. The one or more processors may include graphics processing units (GPUs) and central processing units (CPUs). DIEM determination module 502 may also be implemented in whole or in part via hardware including via field-programmable gate arrays (FPGAs) and application-specific integrated circuits (ASICs).

In one embodiment, server 500 hosts an online portal 504 which may be accessed over network 510 by user computing device 520 executing a web browser 522 to enable a user to determine their current and/or goal diet and its/their associated environmental effects. In another embodiment, user computing device 520 executes an app 524 locally to communicate over network 510 with DIEM determination module 502 in order to determine their diet and its environmental effects. User computing device 520 includes one or more processors 526. Network 510 may be the Internet, a local area network (LAN), intranet, cellular network or some other type of network that enables communication between user computing device 520 and server 500. User computing device may be a smartphone, laptop, tablet, desktop or other type of computing device equipped with one or more processors and a network interface (not shown) enabling communication over network 510.

The exemplary environment may also include one or more additional computing devices 530 executing one or more processors 539 and including one or more specialized databases holding essential information for the DIEM determination process described herein. For example, computing device 530 may include dietary patterns database 531 holding a diet map with information about different diet types and different diet qualities. Computing device 530 may also hold composite image database 532 holding images that are grouped to represent a specific diet type for a specific diet quality over a specified time frame. Similarly, computing device 530 may also hold image vignette database 533 storing image vignettes for photo navigation providing dietary fingerprints of the larger composite image groups. Further, computing device 530 may hold databases for different types of environmental impacts (EI) caused by dietary elements that are needed for DIEM determination including, but not limited to, land use EI dB 535, water use EI dB 536, eutrophication/nitrogen use EI dB 537 and greenhouse gas emission EI db 538. In further embodiments, databases holding EI impacts based on acidification; life cycle analysis; fragile ecosystem impacts; processing and transportation impacts; and more may be utilized. It will be appreciated that although shown separately in FIG. 5, some or all of the depicted databases could be provided via server 500 or divided amongst multiple network accessible computing devices in a manner other than that shown in FIG. 5 without departing from the scope of the present invention.

It should be appreciated the DIEM scoring system for the environmental impact of food choices that is described herein may analyze both horizontally, across component measures (i.e., water use; land use; nitrogen use; greenhouse gas emissions), and vertically from foods to dietary patterns. Combined with the determination of diverse dietary patterns in a map of images, DIEM represents the opportunity for a portal or app that can assess current and/or goal dietary intake for a user, assign an environmental impact score, and immediately compare that score to a range of alternatives. Such easily accessible comparisons may empower and motivate consumers to choose a goal dietary pattern aligned with their tastes and health goals that imposes the minimal environmental footprint.

### Exemplary DIEM System

As a further aid in explanation, an exemplary DIEM system is now described.

In one embodiment, an exemplary system includes environmental impact databases such as the publicly available Harvard T. H. Chan School of Public Health's food-frequency questionnaire environmental database (FFQED) and Heller et al.'s water scarcity footprint database (WSFD). The FFQED includes estimates of a number of environmental impacts including impacts on greenhouse gas emissions and use of high-quality cropland and reactive nitrogen [Nr] (from fertilizer) for 286 distinct constituent dietary elements, as measured from cradle to farmgate and adjusted to account for edible loss from farmgate to table. Water utilization has variable impacts on the environment given the ratio of water demand to water availability (or, conversely, scarcity)-and thus for example the work of Heller et al. adjusts the water impact scores of food items per unit mass with a weighting coefficient that accounts for relative water availability at the site of production. Of note, in the Central Valley of California where a considerable portion of the national food supply is produced, water scarcity is a significant factor. In parts of Florida where citrus fruits are grown, water is abundant. Embodiments give same foods, grown in these two place, different water impact component scores due to variation in water scarcity, and the attendant weighting coefficient. The WSFD accounts for the environmental impact of irrigation water based on regional water scarcity within the U.S. In one embodiment, the water scarcity footprint values from the WSFD may be added to the 286 dietary elements in the FFQED, resulting in a singular food environmental impact dataset that includes greenhouse gas emissions, cropland use, reactive nitrogen use, and water scarcity-adjusted water utilization footprints in their corresponding units of measure: KgCO2eq per gram of food, m2-yr/g, gNr/g, and liters/g, respectively.

The DIEM scoring method illustrates a single cumulative representation of environmental impact, irrespective of unit of measure. In one embodiment, to convert the four unit-specific environmental indicators in the list of 286 foods to unitless scores, Clarke et al.'s environmental impact score calculation method may be utilized (Clark, M., Springmann, M., Rayner, M., Scarborough, P., Hill, J., Tilman, D., Macdiarmid, J. I., Fanzo, J., Bandy, L. and Harrington, R. A., 2022. Estimating the environmental impacts of 57,000 food products. Proceedings of the National Academy of Sciences, 119(33), p.c2120584119). To determine environmental impact, first, the largest impact score within each environmental indicator is identified. Second, the scaled impact for each environmental indicator is found by dividing each score by the largest impact score and then multiplying by 100. Third, the scaled impacts of the environmental indicators are averaged within each dietary element. Fourth, the “composite environmental impact score” is determined by dividing each dietary element's averaged score by the highest averaged score, then multiplying each by 100. To confirm ordinal and incremental precision of data, dietary elements may be divided into the food groupings defined in the FFQED to identify consistency between the composite food group environmental scores and those of previously-published environmental scoring systems.

The 286 dietary elements may be divided into the parent categories that fulfill all food groups for the previously determined dietary patterns. Through a sensitivity analysis, the ranges, medians, and means within each parent category may be compared to identify specific foods that need to be subdivided into separate categories to provide heightened definition. For example, “red meat” may be divided into “beef”, “other red meat”, and “pork”. New categories may be created for “mollusks” and “crustaceans” in contrast to the “fish” parent category. “Cheeses” and “butter” may be individually separated from the “dairy” parent category. “Grains” may be divided into “whole grains” and “refined grains and grain products”. “Corn and olive oil” may be separated from “extracted oils—other”. Vegetables may be divided into “starchy vegetables” and “non-starchy vegetables”. Alcohol may be divided into “liquor”, “beer”, and “wine”. After sub-dividing these parent categories, means, medians, and ranges may be compared to confirm that inter-category variance significantly superseded intra-category variance. This confirmation establishes that parent categories in the system provide the necessary granularity to accurately define food groups that share similar environmental scores while excluding excess detail.

The aggregate environmental impact scores of the dietary elements composing each parent category may then be averaged to determine the parent category aggregate environmental impact score (PCS). The Clarke et al. environmental scoring method may be used to create the raw scores for the parent categories. The highest PCS is identified, each PCS is divided by this score and then multiplied each by 100. This provides an ordinally and incrementally precise method for stratifying all parent categories across the dietary patterns on a 0-100 scale.

An exemplary diet map includes fifty or more dietary patterns and additional patterns may added, ad infinitum, as they are determined. The dietary patterns may be derived from the diverse cultural practices, and the latest scientific literature and range from meat-centric to mixed to plant-based patterns. Representative foods exclusive to the dietary pattern may be stratified per diet quality tier level. The dietary patterns may be analyzed using NDSR (Nutrition Data System for Research) for which nutrient and food group data are available at the food level (Katz D L, Rhee L Q, Katz C S, Aronson D L, Frank G C, Gardner C D, Willett W C, Dansinger M L. Dietary assessment can be based on pattern recognition rather than recall. Med Hypotheses. 2020 Feb. 26;140:109644). Each DIEM parent category (DPC) may be matched to relevant sub food group data. If there is no exact match to available sub food group data, then qualified foods may be determined by inclusion rules. For example, DPC crustaceans may be mapped to sub food group, shellfish. Since shellfish is a broad category that also includes DPC mollusks, an inclusion rule needs to specify shrimp, prawn, lobster, crab, and crayfish. See Table 3 below for an exemplary DPC list. The category matching and inclusion rules allow for a broad application to determined dietary patterns.

TABLE 3

| Parent Food Group Category | Parent Category Score (Mean) |
|---|---|
| beef | 52.3 |
| other red meat | 45.4 |
| extracted oils - corn & olive* | 26.2 |
| processed meat | 26.1 |
| other dairy - butter | 20.6 |
| crustaceans* | 18.8 |
| tree nuts* | 16.7 |
| sweets/dessert items | 9.6 |
| pork | 9.4 |
| cheese | 8.8 |
| poultry | 7.8 |
| fish | 7.2 |
| other dairy | 4.5 |
| alcohol, liquor | 4.3 |
| extracted oils - other* | 3.0 |
| refined grains and grain products | 2.8 |
| eggs | 2.6 |
| mollusks* | 2.5 |
| alcohol, wine | 2.3 |
| whole grains | 2.1 |
| fruits | 1.7 |
| legumes | 1.5 |
| meat alternatives | 1.4 |
| vegetables, starchy | 1.3 |
| beverages | 1.3 |
| vegetables, non-starchy | 1.1 |
| peanuts* | 0.9 |
| dairy alternatives - all | 0.8 |
| alcohol, beer | 0.5 |

*Inclusion rules to apply

In one embodiment, an exemplary Sequence for Food Group (FG) Calculation may be represented as follows:

### Data Collection

1. Nutrient Data
   a. Obtain total energy for each meal/food, including meal/food details such as the nutrient composition of all foods inclusive of macro-and micro-nutrients.
   b. Compile sub-FG totals and total FG.
2. DIEM Data
   a. Retrieve DIEM Food Categories (DFC).
   b. Obtain DFC Environmental Impact (EI) Values.

### Calculations

3. Calculate Sub-FG for Each Meal/Food
   a. Identify the sub-FG for a meal/food per DFC (e.g., Beef).
4. Sum Sub-FG Totals
   a. Calculate the sum of sub-FG totals (e.g., Beef) per 3-day menu.
5. Determine % of Total Calories
   a. Calculate the percentage of total calories of the 3-day meal plan, as a basis to determine the daily mean calorie attribution to each sub-FG represented from sub-FGs based on the total 3-day menu calories.

### Dietary Pattern Application

6. Apply Diet Type/Quality Tier Level
   a. Apply the Diet Type/QT level (3-day menu) to generate raw Total EI value.
   b. Generate the EI value in a display format for the end-user of the app.

In an embodiment a sample FG calculation for diet type per quality tier may be represented as follows:
1. Calculate % of FG Relevant to the 3-day DP
   a. Sum all available Beef calories from the 3-day menu.
   b. Divide by the Total 3-day menu calories.
   c. Multiply by 100 to get the percentage.
2. Calculate Total EI for Beef FG
   a. Obtain Total EI for Beef per QT based on Mean EI.
   b. Divide % Total Beef Calories from Step 1 by 100 to get the ratio.
   c. Multiply by Mean EI for Beef.
3. Repeat steps for each FG
   a. Repeat the above steps for each FG category.
4. Calculate Total % Cal for 3-day DP
   a. Simultaneously calculate the total percentage of calories for the 3-day DP by summing all FGs. This is to see if the majority (˜95%) of the food group calories is accounted for.
5. Calculate Total EI for 3-day DP
   a. Add all EI values from the FGs together.

In one embodiment, lower final calculated scores indicate lower environmental impact. For improved user understanding, scores may be grouped according to deciles and reverse scoring used so that the higher scores indicate which diets are better for the environment.

Exemplary decile ranges for various dietary patterns are depicted in Table 4 below:

TABLE 4

| | Low end | | High end | |
|---|---|---|---|---|
| Deciles | raw score | | raw score | |
| 1 | KET-4 | 10.56428411 | PAL-1 | 21.11856413 |
| 2 | SOU-2 | 8.99857953 | KET-5 | 10.56428411 |
| 3 | SOU-3 | 7.720293903 | LOC-9 | 8.890903611 |
| 4 | -PES 1.00 | 6.499205789 | KOR-5 | 7.650059954 |
| 5 | MED-8 | 5.626163003 | VET-2 | 6.420751916 |
| 6 | VEG-4 | 4.912618319 | FLX-3 | 5.53360099 |
| 7 | VET_CKD-10 | 4.399773116 | -PES 3.00 | 4.894775663 |
| 8 | VET_GF-10 | 3.949414089 | VEG-2 | 4.389600732 |
| 9 | -PES 5.00 | 3.606824226 | LOF-7 | 3.93889063 |
| 10 | WFR-10 | 1.402109013 | FLX-6 | 3.605121432 |

Diet Type Abbreviations
KET = Keto
PAL = Paleo
SOU = Southern American
LOC = Low Carb
PES = Pescatarian
KOR = Korean
MED = Mediterranean
VET = Vegetarian
VEG = Vegan
FLX = Flexitarian
VET_CKD = Vegetarian Chronic Kidney Disease
VET_GF = Vegetarian Gluten Free
LOF = Low Fat
WFR = Whole Food Plant-Based Fat Restricted

### Adjusting Diet Quality Assessment to Account for Cultural Dietary Differences

Embodiments of the present invention extend the ability to assess diet quality

from a culturally agnostic approach to a technique that accounts for cultural diet variances when generating a score for a diet.

There are important limitations to the HEI. The HEI metric is closely aligned with the Dietary Guidelines for Americans, and accordingly confers credit for food groups that prevail in the American diet, including dairy, meat, poultry, fish, seafood, and grains. While allowing for certain diets to substitute legumes as a protein in place of meat, poultry, and fish in the HEI scoring construct, an omission of dairy or grains from a dietary pattern reduces the total, achievable HEI score.

For example, an array of traditional East Asian diets omit dairy. While categorizable as an omission relative to the HEI construct, these East Asian diets in fact never included dairy in the first place. In fact this long-standing inclusion of dairy by select populations, and its exclusion by others, has resulted in marked, demographic variation in the prevalence of lactose tolerance. The native, mammalian condition is lactose intolerance after infancy/weaning, and persistence of lactose tolerance throughout the lifespan represents an adaptation by certain human populations. The East Asian diets are not alone in not including dairy components. Along with select, traditional Asian diets, vegan diets also exclude dairy. Similarly, Paleo diet excludes dairy, and, in many applications, excludes grains as well. This traditional omission becomes important since there is no objective indication that health outcomes, including the most definitive outcomes, vitality and longevity, are adversely affected by the exclusion of dairy when the overall balance of the diet is sound. The same may also be true for the exclusion of grains, although there is less evidence to date. High quality versions of select Asian diets, vegan diets, and potentially Paleo diets, all of which exclude dairy and in some cases grains, are therefore reasonable contenders when considering which dietary patterns are “best”.

Within the United States, which is a multicultural society, there are a wide range of dietary practices, many based on heritage, and others based on alternative nutrition principles and emphasis. This range of dietary practices is of course replicated many time over when considering diets from different parts of the globe beyond the United States. If routinely applied in this context, the standard application of the HEI within a diet quality assessment system is ill-suited to fairly assess diets across this expanse of practices. To address this limitation, generalize the utility of routine diet quality scoring, and provide appropriate credit for overall diet quality across a multicultural expanse of differing assemblies of food groups/components contributing to the whole diet, embodiments introduce an adaptation of the HEI called “adaptive component scoring” in order to fairly account for cultural dietary differences.

To adapt the HEI to dietary patterns that exclude select food groups, the system described herein makes an initial determination about food groups that could reasonably be deemed “discretionary” in a balanced, complete, and sustaining dietary pattern. This determination may be predicated on reviews of pertinent literature, reviews of dietary patterns rooted in heritage that are known to have stood the test of time, and/or the judgment of nutrition and health professionals. Based on the determination, adaptations are made to the standard HEI scoring construct, reproduced as Table 2 below. It should be noted that the HEI-2020 components and scoring standards are the same as HEI-2015. Intakes between the minimum and maximum standards are scored proportionally. The total HEI score is the sum of the adequacy components (i.e. foods to eat more of for good health) and moderation components (i.e. foods to limit for good health).

TABLE 2

| | Maximum | Standard for | Standard for minimum |
|---|---|---|---|
| Component | points | maximum score | score of zero |
| Adequacy: | | | |
| Total Fruits² | 5 | ≥0.8 cup equiv. per 1,000 kcal | No Fruits |
| Whole Fruits³ | 5 | ≥0.4 cup equiv. per 1,000 kcal | No Whole Fruits |
| Total Vegetables⁴ | 5 | ≥1.1 cup equiv. per 1,000 kcal | No Vegetables |
| Greens and Beans⁴ | 5 | ≥0.2 cup equiv. per 1,000 kcal | No Dark Green Vegetables or Legumes |
| Whole Grains | 10 | ≥1.5 oz. equiv. per 1,000 kcal | No Whole Grains |
| Dairy⁵ | 10 | ≥1.3 cup equiv. per 1,000 kcal | No Dairy |
| Total Protein Foods⁶ | 5 | ≥2.5 oz equiv. per 1,000 kcal | No Protein Foods |
| Seafood and Plant | 5 | ≥0.8 oz. equiv. per 1,000 kcal | No Seafood or Plant Proteins |
| Proteins^{5,7} | | | |
| Fatty Acids⁷ | 10 | (PUFAs + MUFAs)/ | (PUFAs + MUFAs)/ |
| | | SFAs ≥ 2.5 | SFAs ≥ 1.2 |
| Moderation: | | | |
| Refined Grains | 10 | ≤1.8 oz equiv. per 1,000 kcal | ≥4.3 oz equiv. per 1,000 kcal |
| Sodium | 10 | ≤1.1 gram per 1,000 kcal | ≥2.0 grams per 1,000 kcal |
| Added Sugars | 10 | ≤6.5% of energy | ≥25% of energy |
| Saturated Fats | 10 | ≤8% of energy | ≥16% of energy |

²Includes 100% fruit juice.
³Includes all forms except juice.
⁴Includes legumes (beans and peas).
⁵Includes all milk products, such as fluid milk, yogurt, and cheese and fortified soy Beverages.
⁶Includes seafood, nuts, seeds, soy products (other than beverages), and beans, peas and lentils.
⁷Ratio of poly- and monosaturated fatty acids (PUFAs and MUFAs) to saturated fatty acids (SFAs).

In one embodiment, adaptive component scoring adjusts the HEI denominator based on the food groups available to contribute “credit” to the numerator. The adapted formula, may be represented as:

Ths=total components (foods and nutrients) in the HEI score
Uhs=universal (required) components in the adapted HEI score
Dhs=discretionary (optional) components in the adapted HEI score

Based on the considerations discussed above, the established categories include:

Uhs: whole fruits; total fruits; seafood & plant protein; greens/beans & total vegetables; nutrient entries (i.e., sat fat; added sugar; sodium; fatty acid ratio [(PUFA+MUFA)/SFA])

Dhs: dairy; whole grains; refined grains; (total protein-seafood&plant protein)

For any given diet, the adjusted scores may be established based on the a priori exclusion of discretionary components, e.g.:

Vegan diets exclude meat, poultry, eggs, fish, and dairy.

Vegetarian diets exclude meat, poultry, and fish.

Asian diets may exclude dairy.

Paleo diets may exclude grains and legumes and dairy

Accordingly:

$$As = Is * ( Ts / ITs )$$

### Where:

As=adjusted HEI score
Is=HEI score for included components
Ts=total possible HEI score for all components
ITs=total possible HEI score for included components

For example:

When a given diet excludes select components and can achieve a maximal score of 80 and an individual's diet within this pattern achieves a score of 60. The adjusted HEI score for this entry is:

$$As = Is * ( Ts / ITs )$$

$$As = 60 * ( 100 / 80 )$$

$$As = 75$$

It should be appreciated that although the example above describes adjusting the denominator, other approaches to adjusting the score may also be employed within the scope of the present invention. For example, rather than lessening the amount of the maximal score, credit for an omitted discretionary component could be added. Such an approach balances out on a relative basis as all diets start with the same credit in such a scenario.

It should be noted that while the current depiction of Adaptive Component Scoring references the Healthy Eating Index, and more specifically the Healthy Eating Index 2020 as the current version of a widely used and robustly validated measure of overall diet quality, the same approach may be applied to future iterations of the Healthy Eating Index, and to any other overall diet quality measure compiled from ‘credit’ for food group/component entries in whole or in part.

It should further be appreciated that since HEI scoring allows for full protein credit from a range of sources not excluded collectively from any balanced diet, namely-meat, poultry, fish and seafood, and plants (i.e., legumes)—identified diets do not require adjustment in this protein area.

The quality of a given dietary pattern derives from the quality of health effects it imparts: disease prevention; health promotion; contributions to vitality and longevity. Invoking such considerations, there is more than one way to achieve a “high quality” diet, and no one culture owns a monopoly on the formula. A universally applicable standard for high diet quality predicated on key health outcomes must allow for cultural variations, including the exclusion of a food group. Adaptive component scoring as described herein respects the fundamental construct of the Healthy Eating Index, while making this crucial accommodation of cultural variation.

Some food groups are clearly discretionary. There are entire human populations that have no long-standing tradition of dairying, for instance, in which lactose intolerance and the exclusion of dairy from the cultural diet both prevail. There are other populations with long exposure to dairy, and obvious adaptation to it as indicated by widespread lactose tolerance, courtesy of a genetic mutation. Of note, both of these groups are represented among the world's “Blue Zones”, famous for their healthy life span. This example illustrates the potential to achieve the same high quality of overall dietary pattern with, and without, dairy. The adaptation of the HEI described herein serves as a quantitative translation of that important principle.

While there are food groups that may be deemed “discretionary” based on modern science, evolutionary biology, and the range of cultural practices, there are clearly food groups that are not. While short-term adjustments might allow for the exclusion of vegetables, fruits, or legumes from the diet, there is no discernible signal across the expanse of evidence sources noted above that such patterns are conducive to optimal health outcomes across the human lifespan. Embodiments are thus directed at those components of an overall dietary pattern that both (a) actually do come and go across an expanse of cultural diversity and prevailing behavior; and (b) can reliably be associated with the same set of health outcomes, summarized as years in life (i.e., longevity), and life and years (i.e., vitality). The combination of vitality and longevity is often referred to as “health span,” meaning the span of healthy life. In practice, this directs the adjustments preferentially to dairy and grains. There is no need to make adjustments for the exclusion of meat, poultry, fish, or seafood not because these don't occur, but because the HEI already accounts for this by allowing for full credit from plant-derived protein sufficient in quantity and quality.

Attention to the diverse means of elevating overall dietary quality for a multicultural society is increasing, but has historically been limited. Among the important implications of this focus is the opportunity to standardize diet quality without standardizing diet type in intervention studies and food-as-medicine initiatives. Familiarity is well established as a key driver of dietary preference, and adherence to prescribed diets predicated on a “one-size-fits-all” approach for a diverse population is known to be rate-limiting in their impacts. An adaptation of the HEI for multicultural deployments offers the promise of innovations in nutrition research and service that could reduce attrition, enhance adherence, improve satisfaction, and generalize far more readily.

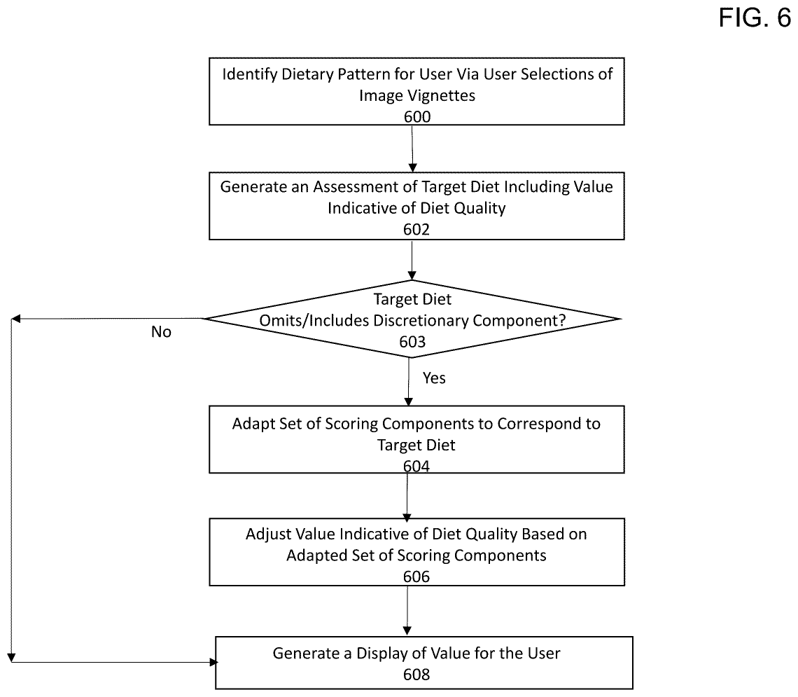

FIG. 6 depicts an exemplary sequence of steps followed by an embodiment of the present invention to determine an impact of cultural dietary differences on an individual's target diet, i.e. the user's current baseline diet, one or more goal diets or both. The sequence begins with a dietary pattern for an individual being identified through the individual's selections of sequentially displayed image vignette choices as explained above (step 600). For example, the individual may be presented with a choice between two or more image vignettes representing dietary choices with each choice leading to a further choice between options until the system identifies the target diet, i.e. the individual's current or goal diet. The system generates an assessment including a value indicative of diet quality based upon the identified dietary pattern (step 602). As part of the assessment, the system determines, based upon pre-determined criteria, if the identified target diet omits any discretionary components (step 603). If there are no discretionary components that have been omitted, a display of the value may be generated for the individual (step 608). Alternatively if there is a discretionary component that has been omitted, the scoring system adapts. First, the assembly of food components contributing to the total diet quality score is adapted to correspond to the target diet (step 604), and secondarily, the resulting, adapted diet quality score is generated based on the new set of contributing food components (step 606). For example, the diet may omit dairy elements which ordinarily form part of the scoring calculation. If the diet does omit a discretionary dietary element, whose absence has been predetermined to not effect health outcomes, the system adjusts the score by recalculating the score without a requirement for that element thereby increasing the value indicative of diet quality (step 606). So for example if a diet value were being generated such that the diet assessment could result in a maximum score of 10 with the dairy component contributing a maximum of 2 to the score but the particular culture's diet does not have dairy elements, the score may be recalculated without the component and would then have a maximum score of 8. Continuing with this example, a diet which attained a score of 6/10 (0.6 or normalized to 6) would have a new value of 6/8 (0.75 or normalized to 7.5). Once the score indicative of diet quality has been adjusted, it may be displayed for the individual (step 606) alone or with additional information about alternative dietary patterns different from the individual's current diet that may lead to better health outcomes. It should be appreciated that the score/value indicative of diet quality may take a number of forms including, without limitation, being displayed in numerical form, displayed as a letter grade or displayed as a color (e.g. green/yellow/red).

It should be appreciated that the adaptive scoring system described herein also encompasses situations where scoring is adapted to credit additional dietary elements being part of a diet that are determined to be beneficial but not initially part of the underlying food scoring system. For example, as the research and evidence regarding the benefits of new dietary elements appears and/or to accommodate newly identified diets, the scoring approach may be expanded to encompass these additional dietary elements.

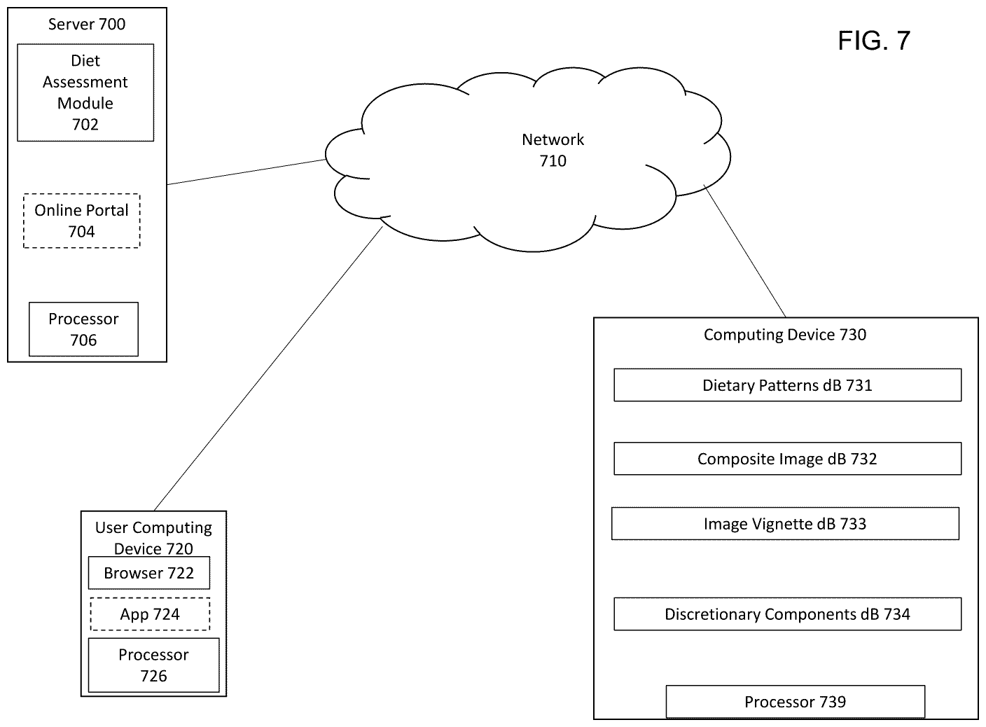

FIG. 7 depicts an exemplary environment suitable for practicing one or more embodiments of the present invention. The exemplary environment, includes one or more servers 700 that are configured to execute a diet assessment module 702 with the aid of one or more processors 706. The diet assessment module 702 may be implemented as one or more software applications configured to perform some or all of the diet assessment sequence discussed in FIG. 6, alone or in parallel. The one or more processors may include graphics processing units (GPUs) and central processing units (CPUs). Diet assessment module 702 may also be implemented in whole or in part via hardware including via field-programmable gate arrays (FPGAs) and application-specific integrated circuits (ASICs).

In one embodiment, server 700 hosts an online portal 704 which may be accessed over network 710 by user computing device 720 executing a web browser 722 to enable an individual to determine their current and/or goal diet and its/their associated environmental effects. In another embodiment, user computing device 720 executes an app 724 locally to communicate over network 710 with diet assessment module 702 in order to determine their diet adjusted for any cultural variance as discussed herein and/or the associated environmental effects. User computing device 720 includes one or more processors 726. Network 710 may be the Internet, a local area network (LAN), intranet, cellular network or some other type of network that enables communication between user computing device 720 and server 700. User computing device may be a smartphone, laptop, tablet, desktop or other type of computing device equipped with one or more processors and a network interface (not shown) enabling communication over network 710.

The exemplary environment may also include one or more additional computing devices 730 executing one or more processors 739 and including one or more databases holding information for the diet assessment process described herein. For example, computing device 730 may include dietary patterns database 731 holding a diet map with information about different diet types and different diet qualities. Computing device 730 may also hold composite image database 732 holding images that are grouped to represent a specific diet type for a specific diet quality over a specified time frame. Similarly, computing device 730 may also hold image vignette database 733 storing image vignettes for photo navigation providing dietary fingerprints of the larger composite image groups. Further, computing device 730 may hold discretionary component database 734 holding information relating to known discretionary food components. It will be appreciated that although shown separately in FIG. 7, some or all of the depicted databases could be provided via server 700 or divided amongst multiple network accessible computing devices in a manner other than that shown in FIG. 7 without departing from the scope of the present invention.

Portions or all of the embodiments of the present invention may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

We claim:

1. A computing device-implemented method for determining dietary impact on environmental measures, the computing device including at least one processor, the method comprising:
providing a database of images depicting a plurality of dietary elements representative of a multi-day dietary intake pattern, the dietary elements being at least one of food and beverages;
receiving, at a server, a request from a client device for images for use in diet determination;
providing, in response to the request, a sequence of image vignettes for photo navigation generated from the database of images, each image vignette generated from a plurality of initial images associated a multi-day dietary intake pattern and including a subset of dietary elements depicted in the plurality of initial images, the subset containing fewer images than depicted in the initial images;
determining a target diet including a diet type and diet quality for a user based on a sequence of photo navigation choices of dietary elements made by the user from among the provided sequence of image vignettes, the determined target diet including a plurality of dietary elements;
determining a plurality of types of environmental impact scores for each of the plurality of dietary elements in the target diet, each environmental impact score respectively associated with a different type of environmental impact and determined using one or more environmental impact databases;
generating a cumulative environmental impact score for each dietary element in the determined diet based on each of the plurality of types of environmental impact scores for that dietary element; and
determining a cumulative environmental impact score for the target diet based on the plurality of generated cumulative environmental impact scores for the plurality of dietary elements in the target diet and a proportional contribution of each dietary element in terms of calories to the target diet.

2. The method of claim 1, wherein the plurality of types of environmental impacts that are determined for each dietary element using the environmental impact databases include a water use impact, a land use impact, a eutrophication/nitrogen use environmental impact and a greenhouse gas emission impact.

3. The method of claim 2 wherein the water use impact is adjusted by an intensity of water use at a site of production for the dietary element.

4. The method of claim 2 wherein the water use impact is adjusted for the use of irrigation water in areas of regional water scarcity.

5. The computing device implemented method of claim 1, wherein determining the target diet further comprises:
determining the user's nutrient intake to establish a habitual calorie level calculated based on a height, a weight, a sex, an age, and an activity level of the user, the activity level determined in part using data collected from wearable device tracking the user's activity level that is communicatively coupled to the client device.

6. The method of claim 1, wherein the target diet is a user's current diet or goal diet.

7. The method of claim 1 wherein the generating of a cumulative environmental impact score for each dietary element in the determined diet is based on a weighted aggregate of the plurality of environmental impact scores for that dietary element.

8. The method of claim 1 wherein the determining of the cumulative environmental impact score for the target diet is based on a weighted aggregate of the plurality of generated cumulative environmental impact scores for the plurality of dietary elements in the target diet.

9. The method of claim 1, further comprising:
determining a cumulative environmental impact score for each of a plurality of target diets; and
ranking the plurality of target diets based on their respective cumulative environmental impact scores to indicate a relative environmental impact among the plurality of target diets.

10. The method of claim 1, wherein the target diet is the user's current diet and further comprising:
generating an assessment of the current diet, the assessment including a value indicative of diet quality;
determining whether the current diet omits at least one discretionary component;
adjusting, when the current diet is determined to include at least one discretionary component, an assessment of the current diet, the adjusting increasing the value indicative of diet quality; and
generating a display of the value indicative of diet quality for the user.

11. The method of claim 10, wherein the assessment is adjusted based on the absence of a dietary component.

12. The method of claim 11, further comprising:
adjusting the assessment based on the absence of dairy components.

13. The method of claim 11, further comprising:
adjusting the assessment based on the absence of grain components.

14. The method of claim 10 wherein the value is displayed as a number, letter or color.

15. A non-transitory medium holding computing device-executable instructions for determining dietary impact on environmental measures, the instructions when executed causing at least one computing device equipped with at least one processor to:
provide a database of images depicting a plurality of dietary elements representative of a multi-day dietary intake pattern, the dietary elements being at least one of food and beverages;
receive, at a server, a request from a client device for images for use in diet determination;
provide, in response to the request, a sequence of image vignettes for photo navigation generated from the database of images, each image vignette generated from a plurality of initial images associated a multi-day dietary intake pattern and including a subset of dietary elements depicted in the plurality of initial images, the subset containing fewer images than depicted in the initial images;
determine a target diet including a diet type and diet quality for a user based on a sequence of photo navigation choices of dietary elements made by the user from among the provided sequence of image vignettes, the determined target diet including a plurality of dietary elements;
determine a plurality of types of environmental impact scores for each of the plurality of dietary elements in the target diet, each environmental impact score respectively associated with a different type of environmental impact and determined using one or more environmental impact databases;
generate a cumulative environmental impact score for each dietary element in the determined diet based on each of the plurality of types of environmental impact scores for that dietary element; and
determine a cumulative environmental impact score for the target diet based on the plurality of generated cumulative environmental impact scores for the plurality of dietary elements in the target diet and a proportional contribution of each dietary element in terms of calories to the target diet.

16. The medium of claim 15, wherein the plurality of types of environmental impacts that are determined for each dietary element using the environmental impact databases include a water use impact, a land use impact, a eutrophication/nitrogen use environmental impact and a greenhouse gas emission impact.

17. The medium of claim 16 wherein the water use impact is adjusted by an intensity of water use at a site of production for the dietary element.

18. The medium of claim 16 wherein the water use impact is adjusted for the use of irrigation water in areas of regional water scarcity.

19. The medium of claim 15, wherein the instructions when executed further:
determine the user's nutrient intake to establish a habitual calorie level calculated based on a height, a weight, a sex, an age, and an activity level of the user, the activity level determined in part using data collected from wearable device tracking the user's activity level that is communicatively coupled to the client device.

20. The medium of claim 19, wherein the target diet is a user's current diet or goal diet.

21. The medium of claim 19 wherein the generating of a cumulative environmental impact score for each dietary element in the determined diet is based on a weighted aggregate of the plurality of environmental impact scores for that dietary element.

22. The medium of claim 15 wherein the determining of the cumulative environmental impact score for the target diet is based on a weighted aggregate of the plurality of generated cumulative environmental impact scores for the plurality of dietary elements in the target diet.

23. The medium of claim 15, wherein the instructions when executed further cause the at least one computing device to:
determine a cumulative environmental impact score for each of a plurality of target diets; and
rank the plurality of target diets based on their respective cumulative environmental impact scores to indicate a relative environmental impact among the plurality of target diets.

24. The medium of claim 15, wherein the target diet is the user's current diet and the instructions when executed further cause the at least one computing device to:
generate an assessment of the current diet, the assessment including a value indicative of diet quality;
determine whether the current diet omits at least one discretionary component;
adjust, when the current diet is determined to include at least one discretionary component, an assessment of the current diet, the adjusting increasing the value indicative of diet quality; and
generate a display of the value indicative of diet quality for the user.

25. The medium of claim 24, wherein the instructions when executed further cause the at least one computing device to:
adjust the assessment based on the absence of dietary components.

26. The medium of claim 25, wherein the instructions when executed further cause the at least one computing device to:
adjust the assessment based on the absence of dairy components.

27. The medium of claim 25, wherein the instructions when executed further cause the at least one computing device to:
adjust the assessment based on the absence of grain components.

28. The medium of claim 23 wherein the value is displayed as a number, letter or color.

29. A system for determining dietary impact on environmental measures, the system comprising:
one or more databases of images depicting a plurality of dietary elements representative of a multi-day dietary intake pattern, the dietary elements being at least one of food and beverages;
one or more environmental impact databases holding measurements of a plurality of types of environmental impacts associated with the plurality of dietary elements, and
a server configured to receive a request from a client device for images for use in diet determination, wherein in response to the request:
a sequence of image vignettes for photo navigation generated from the database of images is provided, each image vignette generated from a plurality of initial images associated a multi-day dietary intake pattern and including a subset of dietary elements depicted in the plurality of initial images, the subset containing fewer images than depicted in the initial images,
a target diet that includes a diet type and diet quality is determined for a user based on a sequence of photo navigation choices of dietary elements made by the user from among the provided sequence of image vignettes, the determined target diet including a plurality of dietary elements,
a plurality of types of environmental impact scores for each of the plurality of dietary elements in the target diet is determined, each environmental impact score respectively associated with a different type of environmental impact and determined using the one or more environmental impact databases,
a cumulative environmental impact score for each dietary element in the determined diet is determined based on each of the plurality of types of environmental impact scores for that dietary element, and
a cumulative environmental impact score for the target diet is determined based on the plurality of generated cumulative environmental impact scores for the plurality of dietary elements in the target diet and a proportional contribution of each dietary element in terms of calories to the target diet.

30. The system of claim 29, wherein the plurality of types of environmental impacts that are determined for each dietary element using the environmental impact databases include a water use impact, a land use impact, a eutrophication/nitrogen use environmental impact and a greenhouse gas emission impact.

31. The system of claim 30 wherein the water use impact is adjusted by an intensity of water use at a site of production for the dietary element.

32. The system of claim 30 wherein the water use impact is adjusted for the use of irrigation water in areas of regional water scarcity.

33. The system of claim 29, further comprising:
a wearable device communicatively coupled to the client device that is configured to collect data of the user's activity level, wherein the activity level is used along with a height, a weight, a sex, and an age of the user to determine a nutrient intake to establish a habitual calorie level of the user to determine the target diet.

34. The system of claim 29, wherein the target diet is the user's current diet and the determining of the target diet further:
generates an assessment of the current diet, the assessment including a value indicative of diet quality;
determines whether the current diet omits at least one discretionary component;
adjusts, when the current diet is determined to include at least one discretionary component, an assessment of the current diet, the adjusting increasing the value indicative of diet quality; and
generates a display of the value indicative of diet quality for the user.

35. The system of claim 34, wherein the assessment is adjusted based on the absence of a dietary component.

36. The system of claim 35, wherein the assessment is adjusted based on the absence of dairy components.

37. The system of claim 35, wherein the assessment is adjusted based on the absence of grain components.

38. The system of claim 29, wherein the value is displayed as a number, letter or color.
